(19) United States
(12) Patent Application Publication
(10) Pub. No.: US 2024/0423928 A1
(43) Pub. Date: Dec. 26, 2024

(54) Pharmaceutical Composition and Method for Treating Neurodegenerative Disorders Utilising the Pharmaceutical Composition

(71) Applicant: TAIPEI MEDICAL UNIVERSITY, Taipei City (TW)

(72) Inventors: Chi-Yuan CHIU, Taipei City (TW); Chien-Tai HONG, New Taipei City (TW); Chaur-Jong HU, Taipei City (TW); Chien-Chung CHEN, Taipei City (TW); Shu-Mei CHEN, New Taipei City (TW); Lung CHAN, New Taipei City (TW); Wan-Ting HUANG, Taipei City (TW); Chee-Ho CHEW, New Taipei City (TW)

(21) Appl. No.: 18/746,300

(22) Filed: Jun. 18, 2024

Related U.S. Application Data
(60) Provisional application No. 63/522,027, filed on Jun. 20, 2023.

(51) Int. Cl. A61K 9/70; A61K 35/13; A61K 35/28; A61K 47/34; A61P 25/28

(52) U.S. Cl. CPC A61K 9/7007; A61K 35/13; A61K 35/28; A61K 47/34; A61P 25/28

(57) ABSTRACT

A pharmaceutical composition for treating neurodegenerative diseases of the brain is provided and including cells encapsulated by microtubular array membranes (MTAMs). The material of the microtubular array membrane is Polysulfone (PSF) or PLGA-PLLA copolymer. The cells include hybridoma cells capable of secreting anti-Tau antibodies or human umbilical cord mesenchymal stem cells (hUC-MSCs). The pharmaceutical composition could be implanted into a living body for treating neurodegenerative diseases of the brain in a subject in need thereof, in order to improve the cognitive dysfunction.

### RELATED APPLICATIONS

The present application claims priority to provisional application No. 63/522,027, filed on Jane 20, 2023, the content of which is incorporated herein by reference in its entirety.

### FIELD OF THE INVENTION

The present disclosure relates to an application of Microtube Array Membrane (MTAM) for drug encapsulation, and more particularly, relates to a pharmaceutical composition for treating neurodegenerative diseases of the brain.

### BACKGROUND OF THE INVENTION

Alzheimer's disease (AD) is a well-known neurodegenerative disease. The status of a healthy brain gradually deteriorates over time. Symptoms of Alzheimer's disease such as dementia start interfering with one's basic activity and memory in their life span when the disease progress into mild cognitive impairment. Statistics show an astonishing prevalence of Alzheimer's disease in the United States. It was estimated that 5.8 million Americans of all ages had AD dementia in 2019. Among this population, 81% of them were elders over 75 years old. Thus, AD impacted tremendously on the economic burden. It cost about $290 billion in public health care in the USA in 2019.

Multiple factors contribute to the development of AD, including, but not limited to, brain tumors and deficiency in vitamin D or B12. Scholars have gradually disclosed the relationship between amyloid-β (Aβ) and tau protein. They suggest that tau protein and Aβ were disposed to develop simultaneously. On the other hand, these neurotoxic proteins cause synapse loss, oxidative stress, mitochondrial dysfunction, and neuronal death, eventually, accompanying cognitive decline.

Genetically, the early onset of AD was derived from a hereditary disease. There are three main autosomal dominant mutated genes: APP, PSEN1, and PSEN2. These genes guided an increase in Aβ42:Aβ40 ratio, either a rise in Aβ42 and decline in Aβ40, or an increase in both. As a result, the pathway “amyloidogenic cascade” was unlocked. For late onset cases, Apolipoprotein E (ApoE) and apoE4 isoforms have been identified to be responsible for a late onset of AD.

Among the multitude of therapies advancement, encapsulated cell therapy (ECT) is one of the leading proposed strategies for the treatment of AD. The fundamental concept of encapsulated cell therapy derived from the 1950s. This technique encapsulates functional living cells within a semi-permeable membrane. This technique prevents encapsulated cells from coming into contact with the adjacent host's tissue. This in turn, eliminates the possibility of triggering an immune response which might lead to rejection. The porous membrane confers sufficient protection while ensuring supply of oxygen and nutrients diffuse through for cell proliferation, migration, and differentiation. These functional or genetically engineered cells enclosed in the membrane can steadily produce therapeutic substances and specific cellular secretions.

In one example, genetically engineered C2C12 mouse myoblasts were used to release murinized IgG2a antibodies against Aβ oligomers and plaques based on the amyloid cascade hypothesis. C2C12 myoblasts inserted in a PEG hydrogel device (10⁶ cells). In the AD mice model, after subcutaneous implantation (TauPS2APP mice, 5×FAD mice), IgG2a was continually secreted by the cells to aid in the clearance of Aβ plaques and to prevent additional tauopathy and tau phosphorylation. In another example, PLGA with curcumin nanoparticles prevents Aβ buildup; however, if negative effects occur, reversing them is impossible.

Recently, there were numerous immunotherapies for anti-Aβ and tau proceed in clinical trials. For example, DC8E8 antibodies inhibit tau-tau interaction by masking the neuron surface proteoglycans. Besides, introducing a method to halt the early progress of AD pathology in clinical trials, for example, there were compounds amid at halting the formation of amyloid precursor protein (APP) and interfering with the production of Aβ peptide. Inhibitors of γ-secretase “LY450139 (semagacestat)” were developed in Phase III trial (new therapy vs standard existing therapy): however, it not only alleviates the progress of the disease, but also deteriorate cognitive function. Additionally, the compound “methylene blue (chloride methylthioninium: Rember™, TauRx Therapeutics, Singapore)” completing a Phase II trial (safety and effectiveness), demonstrated the ability to dissolve paired helical filaments (PHFs) of hyperphosphorylated tau, thereby hindering tau aggregation. It showed improvement of cognitive dysfunction in a behavior test by using a tau-transgenic animal model.

Despite the positive results discussed above, ECT systems often fall into one of two categories: macroscale systems with a small diffusion surface area or microscale systems with a large diffusion surface area. As a result, current ECT systems are either macroscale, which are recoverable but have a long diffusion distance from the surface to the cells housed within and a low effective surface area, or micron scale, which are nonrecoverable but have a short diffusion distance (50 microns) and an excellent, effective large surface for the diffusion of nutrients, gases, and the target therapeutic product. As a result, in present techniques, the inability to recover the encapsulated cells if the treatment has unfavorable/side effects is a big stumbling barrier. The ability to retrieve ECT equipment would be critical in future therapeutic applications, as it would increase patient biosafety.

### SUMMARY OF THE INVENTION

Microtube Array Membrane (MTAM) are made up of one-to-one coupled ultra-thin microtube fibers that are organized in an array. The lumen walls of MTAMs are 100 times thinner than those of standard hollow fibers (HFs) as compared to traditional HFs. Furthermore, the capacity to alter the microstructures of MTAMs enables to use in a variety of applications, including but not limited to encapsulated cell therapy, anti-cancer drug screening, tissue regeneration, green energy, fermentation, bioreactors, and so on.

Based on the above objects, the present disclosure provides a pharmaceutical composition for treating neurodegenerative diseases of the brain.

Based on the above objects, the present disclosure further provides the use of an encapsulated cell for preparing a pharmaceutical composition for treating neurodegenerative diseases of the brain. The pharmaceutical composition comprises the encapsulation of cells within MTAMs, where the MTAMs is made of Polysulfone (PSF) or a copolymer of Poly(lactic-co-glycolic acid) and Poly(-L-lactic acid) (PLGA-PLLA). The cells comprise hybridoma cells capable of secreting anti-Tau antibodies or stem cells. The stem cells comprise Neural stem cells (NSCs), umbilical stem cell, Amniotic fluid stem cells (AFSCs), bone stem cells or adipose stem cells. The stem cells comprise human umbilical cord mesenchymal stem cells (hUC-MSC).

Based on the above objects, the present disclosure further provides a treatment of neurodegenerative disorders of the brain utilizing the pharmaceutical composition, comprising implanting the aforementioned pharmaceutical composition in a living body.

The encapsulation of hybridoma within Polysulfone (PSF) MTAMs serves as a potential ‘middle path’ ECT solution, offering tremendous value to future patients by incorporating the ability to be recoverable in the event of side effects. According to common knowledge, if the diffusion distance exceeds 50 microns, nutrients and other substance cannot effectively diffuse. However, the diffusion distance of MTAMs is at most only 30 microns, which enhances the transfer efficiency of nutrients and substances. When the aforementioned general aspects are paired with its outstanding biocompatibility and trans lumen wall diffusion, it becomes a potentially attractive platform to investigate in ECT systems as a prospective faster, accurate, and convenient effect on AD treatment

### BRIEF DESCRIPTION OF THE DRAWINGS

FIG. 1A to FIG. 1F are transverse views of the Scanning ElectronMicroscopy (SEM) images of electrospun PSF MTAMs at different magnification (50×, 200×, 500×, 1000×, 5000× and 10,000×). FIG. 1G and FIG. 1H are top views of the SEM images at a magnification 1000× and 5000×, respectively.

FIG. 1I to FIG. 1K show the distribution of the MTAMs length, width and pore size.

FIG. 1L shows the optical microscopy images of the hybridoma cells cultured on TCPs, as reference. FIG. 1M to FIG. 1N show the optical microscopy images of the PSF MTAMs loaded with hybridoma. FIG. 1O shows the distribution size of the hybridoma that were loaded in the PSF MTAMs.

FIG. 2A to FIG. 2L show the fluorescence microscopy images (4× magnification) of Live-Dead stain of hybridoma cells loaded within PSF MTAMs at day 1 (FIG. 2A to FIG. 2C), day 3 (FIG. 2D to FIG. 2F), day 5 (FIG. 2G to FIG. 21) and day 7 (FIG. 2J to FIG. 2L).

FIG. 2M is a total fluorescence density of hybridoma cells loaded within the PSF MTAMs from day 1 to day 7. FIG. 2N shows the overall viability of hybridoma cells assayed with MTT assay.

FIG. 3A shows the in vitro cell viability of hybridoma cells loaded within PSF MTAMs quantified via MTT assay. FIG. 3B is the concentration of IgG2b antibody detected within the culture medium of various culture settings at day 21.

FIG. 3C shows the immunoreactivity in the pyramidal layer of the hippocampus in human tau (IN4R) transgenic mice to the hybridoma supernatant (anti-tau IgG2b). Arrows points out the strong immunoreactivity in neurons and neurites. Scale: 50 μm. FIG. 3D shows the immunoblotting of the hybridoma supernatant (anti-tau IgG2b) on different protein samples. Molecular weight (arrows) is given on the left of the blot. FIG. 3E shows the images of the implanted PSF MTAMs loaded with hybridoma cells. FIG. 3F shows the in vivo assay of the IgG2b antibody at day 90. Animal model with PSF MTAMs loaded with hybridoma cells registered a concentration of 0.25±0.07 mg/mL, which was statistically significantly greater than the animal model with the empty PSF MTAMs.

FIG. 4A to FIG. 4D is the Morris water maze escape latency of the respective models. FIG. 4A is a design of the Morris water maze. FIG. 4B is the escape latency of the respective mouse model study groups. FIG. 4C shows the travel time in the goal quadrant (long term memory testing). FIG. 4D shows the travel time in the goal quadrant (short term memory testing).

FIG. 5 shows the passive avoidance test mouse models of day 1 to day 3 (baseline) and day 46 of the respective study groups.

FIG. 6A shows the immunohistochemistry of hybridoma cell loaded PSF MTAMs. FIG. 6B shows the immunohistochemistry of the empty PSF MTAMs. FIG. 6C to FIG. 6E is a tissue section of the brain of mouse models. IgG2b antibodies appeared to be brown within nucleus, and blue when not within nucleus (counterstained by hematoxylin dye).

FIG. 6F to FIG. 6H show the percentage of IgG2b positive signal in individual cells (y-axis) versus hematoxylin signals (x-axis). FIG. 6I to FIG. 6K show the percentage of IgG2b positive signal in individual cells within each IgG2b positive area.

FIG. 6L is a graph depicting the IgG2b positive signal in a single cell. FIG. 6M shows the corresponding IgG2b antibody distribution in total area.

FIG. 7A to FIG. 7C show tissue sections of the brain of mouse models of wild-type C57BL/6J mice without treatment (n=5), Triple-transgenic (3×Tg) mice with empty PSF MTAMs (IN: n=3), and Triple-transgenic (3×Tg) mice with hybridoma cell loaded PSF MTAMs (IN; n=4) along with the corresponding magnified sections. P-Tau (detected via Ser199/Ser202 antibodies) appeared to be brown in color.

FIG. 7D to FIG. 7F show the percentage of P-Tau positive signal in a single cell (y-axis) with hematoxylin signal (x-axis). FIG. 7G to FIG. 7I show the percentages of P-Tau positive signal in a single cell versus P-Tau positive area.

FIG. 7J is a graph depicting P-Tau positive signal in a single cell. FIG. 7K shows the corresponding IgG2b antibody distribution in total area. FIG. 7L shows the total Tau levels of the respective study groups.

FIG. 8A shows the western blot analysis of the respective study groups of the cortex and hippocampus regions targeting to P-Tau protein (79 kDa) and beta-actin (49 kDa). FIG. 8B shows the ratio of the P-Tau protein (79 kDa) after normalization by loading control.

FIG. 9A shows the transverse views of the SEM images of PLGA-PLLA MTAMs (magnification 200×). FIG. 9B is the surface pore diameter of PLGA-PLLA MTAMs (magnification 5000×).

FIG. 10A to FIG. 10C shows the cell viability of human umbilical cord-derived mesenchymal stem cell (hUC-MSC) loaded within PSF MTAMs or PLGA-PLLA MTAMs via MTT assay. FIG. 10A shows the cell viability of hUC-MSC loaded within PSF MTAMs. FIG. 10B shows the cell viability of hUC-MSC loaded within PLGA-PLLA MTAMs. FIG. 10C shows the standardization of OD values from day 1 (100%).

FIG. 11 shows the in vivo cell viability of hUC-MSC loaded within PLGA-PLLA MTAMs.

FIG. 12 shows the in vivo cell viability of hUC-MSC loaded within PLGA-PLLA MTAMs (at day 28).

FIG. 13A to FIG. 13C show the Nanoparticle Tracking Analysis (NTA) on hUC-MSC. FIG. 13A shows the size distribution of released particles representing each time point. FIG. 13B shows the mean of the highest diameter at 90% (D90 nm) within samples representing each time point. FIG. 13C shows the concentration of particles representing each time point.

FIG. 14A to FIG. 14B show the expression of the p-TAU protein in Alzheimer's disease (AD) cells after co-culture with hUC-MSC encapsulated in MTAMs. FIG. 14A is a graph depicting the pTAU ELISA assay conducted after co-culturing AD cells with hUC-MSCs. FIG. 14B is the experimental procedure.

FIG. 15 shows the evaluation of memory function in Mitopark mice (an animal model of Parkinson's disease, PD) via novel object recognition (NOR) test at 0, 1, 2, and 3 months after MTAMs implantation.

FIG. 16A to FIG. 16B show the evaluation of memory function in APP/PSI mice (an animal model of Alzheimer's disease) via NOR test after 3 months of MSC (MTAM) treatment. FIG. 16A is the representative movement traces of relative models recorded within 10 minutes. FIG. 16B depicts the percentage of time spent on the novel object. Data are presented as mean±SEM. The tracks and data were obtained using Etho Vision XT.

### DETAILED DESCRIPTION OF THE PREFERRED EMBODIMENTS

In an embodiment of the present disclosure, provided is a pharmaceutical composition for treating neurodegenerative diseases of the brain. The pharmaceutical composition comprises cells encapsulated by microtubular array membranes (MTAMs), the cells comprise hybridoma cells capable of secreting anti-Tau antibodies or human umbilical cord mesenchymal stem cells (hUC-MSCs).

The material of the microtubular array membrane is polysulfone (PSF) or a copolymer of poly(lactic-co-glycolic acid) and poly-L-lactic acid (PLGA-PLLA copolymer). In the case which the material is PLGA-PLLA, the PLGA and PLLA have the same weight in the total weight of the PLGA-PLLA contained in the microtubular array membrane. Specifically, the fibers of the microtubular array membrane are arranged in a monolayer array along an extension direction. The microtubular array membrane is a highly aligned and densely packed electrospun fibers assembly, in which the fibers form a monolayer membrane, and the orientation of the fibers relative to the longitudinal axis of the assembly is not greater than +/−5°.

The wall thickness of a single lumen of the microtubular array membrane is in the range of 3.5 μm to 8 μm. Preferably, the wall thickness can be about 4 μm, 4.5 μm, 5 μm, 5.5 μm, 6 μm, 6.5 μm, 7 μm, 7.5 μm, or 8 μm.

The average lumen size of the microtubular array membrane is 80 μm to 120 μm in height and 40 μm to 60 μm in width. Preferably, the height is in the range of 80 μm to 120 μm and the width is in the range of 25 μm to 45 μm. The height can be about 85 μm, 90 μm, 95 μm, 100 μm, 105 μm, 110 μm, 115 μm, or 120 μm. The width can be about 30 μm, 32 μm, 35 μm, 40 μm, or 42 μm.

The pore size of the microtubular array membrane is in the range of 100 nm to 900 nm. Preferably, the pore size is in the range of 120 nm to 800 nm. The pore size can be about 130 nm, 150 nm, 160 nm, 180 nm, 200 nm, 230 nm, 250 nm, 300 nm, 400 nm, 500 nm, 600 nm, 700 nm, or 800 nm.

Specifically, the type of stem cell is a pluripotent stem cell (PSCs), an induced pluripotent stem cell (iPSCs), a multipotent stem cell, or a lineage-restricted stem cell. The stem cell can be an adult stem cell. In one specific embodiment, the stem cell is a mesenchymal stem cell. The source of the stem cell can be neural stem cells (NSCs), umbilical cord stem cells, amniotic fluid stem cells (AFSCs), bone stem cells, adipose stem cells, or umbilical cord tissue-derived mesenchymal stem cells (hUC-MSCs), but not limited thereto. The content related to the aforementioned stem cells is cited from document.

The average diameter of the cells encapsulated in the microtubular array membrane is in the range of 12 μm to 18 μm. Specifically, the average diameter of the cells can be about 13 μm, 14 μm, 15 μm, 16 μm, or 17 μm.

In one embodiment, the present disclosure provides a use of an encapsulated cell for preparing pharmaceutical composition for treating neurodegenerative diseases of the brain, wherein the pharmaceutical composition is the aforementioned pharmaceutical composition, which is implanted intracranially (IN) or subcutaneously (SC) into a living body. However, the implantation methods are not limited thereto, and as long as the extracellular vesicles can release the microtubular array membrane, the purpose of the present invention can be achieved.

In one embodiment, the present disclosure provides a method for treating neurodegenerative diseases of the brain to a subject in need thereof, comprising implanting the aforementioned pharmaceutical composition into a living body. The implantation methods include intracranial (IN) implantation or subcutaneous (SC) implantation. However, the implantation methods are not limited thereto, and as long as the extracellular vesicles can release the microtubular array membrane, the purpose of the present invention can be achieved. The implantation site can be determined based on the desired site to be improved or/and the treatment range affected by the release area of the extracellular vesicles. The aforementioned neurodegenerative diseases include Alzheimer's disease or Parkinson's disease, but are not limited thereto.

The present disclosure provides the following specific embodiments to deepen comprehension of its spirit.

### Materials and Methods

### 1. Elecrospining of PSF MTAMs

PSF beads (Sigma-Aldrich, Taipei, Taiwan) and polyethylene glycol (Sigma-Aldrich, Taipei, Taiwan) were mixed until homogenous in a 7:3 mixture of N,N-dimethyl formamide (DMF) and dichloromethane (DCM). Under ambient conditions, the resulting polymer solution was electrospun as a ‘shell solution’ with a ‘core solution’ comprised of polyethylene glycol (Sigma-Aldrich, St. Louis, MO, USA) and polyethylene oxide (Sigma-Aldrich, St. Louis, MO, USA). The high voltage charge supply (You-ShangCo., Fongshan City, Taiwan) generated electrostatic force was set up at 4.5-7 kV with a current sustained at 750 μA. The distance between the stainless-steel Co-axial spinneret and the rotating drum collector was 50 mm. The rotating drum collector spun at the speed of 100±10 rpm (radius: 70 mm, 0.73±0.07 m/s). After that, the PSF MTAMs were extracted and washed in double distilled water (ddH₂O), then air-dried. Using a scanning electron microscope, the microstructure characteristics of the PSF MTAMs were measured (SEM: Hitachi, Chiyoda City, Japan).

### 2. PSF MTAMs as a Culture Substrate for Hybridoma Cells.

Mice were immunized with recombinant human Tau protein as previously described. Briefly, cells from the spleen were fused with myeloma cells to obtain an hybridoma that produced antibodies. Anti-Tau antibody secreting hybridoma cells were isolated to obtain a single clone which was amplified. Hybridoma supernatant contains a monoclonal anti-tau IgG2b antibody which recognizes all tau proteins by immunoblotting and neurofibrillary tangles in tau transgenic mice by immunohistochemistry. In the embodiment of the present disclosure, the respective hybridoma cells were centrifuged at 1200 rpm for 5 minutes in 50 mL conical tubes. The supernatant was discarded. The pellets were collected and suspended in DMEM media at a density of 2×10⁵ cells/10 μL. After that, the relevant PSF MTAMs were used to siphon 10 μL of cell suspension that had been pre-sterilized with UV radiation and diced into 0.5 cm×2.0 cm pieces.

In order to prepare culture medium, 85% DMEM-high glucose (which contained 4500 mg/L glucose and L-glutamine, 3.7 g sodium bicarbonate), 15% FBS (HyClone characterized Fetal Bovine Serum, U.S. Origin) and 1% PSA (Penicillin/Streptomycin/Amphotericin B: GeneDireX, Inc., New Delhi, India) were prepared and mixed accordingly. Next, hybridoma cells were suspended in freshly prepared medium and incubated at 37° C. in a standard 75T flask, and electrospun PSF MTAMs that were precut into dimensions of 0.5 cm×2.0 cm under 5% CO₂ atmosphere. After the predetermined duration, the respective hybridoma were retrieved and centrifuged at 250×g for 5 minutes to separate the supernatant which was used for antibody quantification and the pallet for cell viability assessment via MTT assay (3-(4,5-dimethylthiazol-2-yl)-2,5-diphenyltetrazolium bromide).

At the predetermined time points, the respective hybridoma cell loaded PSF MTAMs were retrieved and stained with calcien-AM (Live cells: Biolegend, San Diego, CA, USA) and propidium iodide (Dead cells: Biolegend), incubated for an hour in the dark and immediately visualized under a fluorescent microscope.

### 3. IgG2b Antibody Quantification.

In the antibody quantification, the supernatant from the abovementioned first centrifugation was transferred into a Amicon® Ultra-15 (30 kDa) Centrifugal Filter (tube) and centrifuged at 7500×g at 25° C. for 15 minutes. The concentrated antibodies were suspended accordingly. Next, 8 points of standard twice diluted in series with standard diluent (the last is blank) were prepared. In each well of the plate, 100 μL standard and samples were added, and the plate was secured with a plate sealer, which was followed by an hour of incubation at 37° C. Any excessive liquids were removed and 100 μL of biotin-conjugated IgG2b antibody (Detection Reagent A) was added and re-incubated for an hour at 37° C. This process was followed by a wash with a 0.05% Tween-20 in PBS and then decanting and drying. Next, 100 μL of avidin conjugated to Horseradish Peroxidase (HRP) (Detection Reagent B) was added and incubated for 30 minutes at 37° C. Finally, 90 μL TMB substrate was added to each well (turn blue) and left for a 10-20 minutes reaction time. The reaction was terminated by adding 50 μL stop solution (sulfuric acid) to each well and assayed at 450 nm wavelength in an ELISA reader.

### 4. In Vivo Assay.

### 4.1 Mice Models and Implant of PSF MTAMs.

In this section, Wild-type C57BL/6J mice (female, 2 months old) and Triple-transgenic (3×Tg: female, 2 months old) mice inherited the PSIM146V, AβPPswe and tauP301L transgenes (Jackson Laboratory, Bar Harbor, ME, USA). All the mice were housed in the standard cage and regularly fed on water and food under temperature and humidity-controlled with the 12 hours. light/12 hours dark cycle. All the protocols, procedures, and surgery on the animal were accepted by the Institutional Animal Care and Use Committee or Panel (IACUC/IACUP) of Taipei Medical University. Approval No: LAC-2021-0027.

The hybridoma cells were grown as described above before being siphoned into the PSF MTAMs at a cell density of 2×10⁵ cells/10 μL and the ends impulse sealed. The cell-loaded PSF MTAMs were then grown in outlined DMEM medium for 24 hours at 37° C. in a 5% CO₂ environment. For subcutaneous (SC) implantations, the respective mice were anesthetized with xylazine/zoletil mixture (1:1) diluted 10 times by ddH₂O and administrated through intraperitoneal (IP) injection. The mice's back fur was shaved and sterilized with 75% alcohol. Next, 0.5 cm longitudinal incisions were made as well as separated mucus layer underlying flesh by surgery scissors. Cell loaded MTAMs were well-placed on laboratory spatulas and then implanted subcutaneously. The incisions were sutured and the mice were laid on the warm pad until the body temperature recovery before being returned to their respective housings. In the case of intracranial (IN) implantation, the procedure involved were modified based on works by other reference. Briefly, the respective hemisphere of the brain of the mouse models were subjected to craniotomy (three alternating swipes of

70% alcohol and betadine were perform within the surgery). Minimal dura matter was excised and PSF MTAMs were implanted at the site of excision. The respective bone plates were replaced and secured, and the wound site was closed with sutures.

### 4.2 Assessment/Indicators of the Impact of the Implantation of PSF MTAMs in Mice Models

### Animal Behavior Test

### Morris Water Maze (MWM)

A circular pool with white bottom measuring 150 cm in diameter with a depth of 50 cm was prepared accordingly. Next, 4 high contrast spatial signs were marker on the respective sections of the pool wall (East, West, North, and South). A platform was placed in the target quadrants at a height of 1.5 cm above the water line. Memory acquisition training was carried out by transferring preadapted mouse models into the respective regions of the pools, and directional guidance toward the platform was provided (in cases where the mouse models were unable to find the platform within a minute). The mice were allowed to stay on the platform for 15 seconds. Between day 2-5, the pool was immersed with milk powder to a height of 2 cm above the platform. Mouse models were given 1 minute to find the platform. By day 6, the platform was removed, and similar to the steps outlined during the training sessions, the respective mouse models were given 2 minutes to explore the pool without the platform attached. All the parameters were recorded and analyzed by ActualTrack: Animal Behavior Analysis Software (A-M System, Sequim, WA, USA).

### Passive Avoidance Test

A passive avoidance box (PACS_091120, Columbus Instrument) had 2 compartments (light and dark) connecting with a passage was procured. Mice of all study groups were allowed to separately adapt to the environment within the box on day 1 for at least 30 minutes. Next, the respective mouse models were transferred into the dark region of the box (gate closed) and allowed to explore for 5 minutes. This process was repeated with the light region and the respective mouse models were allowed to explore for 30 seconds, and followed by turning on the lighting with the gate raised. The gate was lowered after the mouse models moved toward the dark region of the box and allowed to stay for 30 seconds before being physically removed and returned to their respective housings. After 30 minutes in their respective housings, the respective mouse models were transferred back into the box, and they were subjected to an electric shock in the foot (0.5 mA for 10 seconds) every time they entered the dark component. On the probing days (day 2-day 3), the respective mouse models were placed in the light component and left to explore for 270 seconds: with the following process similar to those outlined in day 1, with the absence of electric foot shock. This process was continued for the entire 1.5 months duration and any changes was noted accordingly.

### Immunohistochemistry and Brain Tissue Section.

To assess the formation and property (IgG2b expression) of the hybridoma cell growing environment in the PSF MTAMs, the investigation of the timeline was prolonged to 2 months. Each mouse was carefully anesthetized with xylazine/zoletil mixture (1:1) diluted 10 times by ddH₂O via IP and followed by transcardial perfusion with 0.9% NaCl (normal saline). The respective mouse models were sacrificed according to the approved procedure of IACUC/IACUP of Taipei Medical University. Next, the mice brain tissues were dissected into 2 halves and one of the halves was immersed into 10% neutral buffered formalin for 1 day at 4° C. After fixation, the brain samples were sent to the CIS-biotechnology (Taichung, Taiwan) to proceed with paraffin-embedded, slicing into histological slides. Briefly, for paraffin-embedded brain tissue, the brain tissues were dehydrated by immersing them in gradually increasing concentration of ethanol (70-100%) followed by pure xylene. The dehydrated tissues were embedded in a 60° C. melted paraffin. The brain tissues sections were cut to a thickness of 4 μm by microtome before being stored at room temperature.

When immunohistochemistry staining is ready to be carried out, tissue slides were deparaffinized and rehydrated by first in xylene and gradually diluted ethanol (95-50%). The tissue slides were placed in a plastic rack with antigen-retrieval buffer (CIS-BIO, D3316). The rack was transferred into a 95° C. water bath for 10 minutes and was left to cool. The respective slides were washed with 0.025% Triton X-100 in Tris-Buffered Saline (TBS) (Tris Base, NaCl and pH 7.6). Next, slides were blocked in serum and 1% BSA in TBS for 2 hours. at room temperature (RT), and then washed with 0.025% Triton X-100 in TBS. After the tissue slides were treated with 0.3% H₂O₂ in TBS for 15 minutes to inactivate endogenous peroxidase activity, which caused high background, Dako REAL™ EnVision™ Detection System (EnVision) secondary antibody (K5007) (Labeled Polymer) was applied for 30 minutes. The tissue slides were then rinsed with TBS. The IgG2b expression was visualized by adding the substrate, Dako REAL™ EnVision™ Detection System (3,3′-diaminobenzidine, DAB) for 3 minutes (2 times). Before visualizing, the tissue slides were counterstained by Hematoxy lin-Eosin (H&E) Staining Kit—for paraffin sections (CIS biotechnology M700, Taipei, Taiwan). When this step was completed, tissue slides were air-dried and applied Leica micromount, (type 3801731) with the coverslip.

### Western Blot

Fresh sample of brain tissues from the mouse models were snap freeze and store at −80° C. before being used. For protein extraction, prepare tissue lysate samples by adding tissue lysis buffer (RIPA buffer) (0.05 M Tris, pH 8.0:0.005 M EDTA, 0.15 M NaCl, 0.5% SDS, 1% Triton 100X+1 tablet of protease inhibitor), followed by 1-time freeze and thaw, and 3-time centrifugation at 4° C. at 15,000 rpm for 15 minutes. The resulting supernatant was collected, and the protein quantification was carried out via Bradford protein assay. The entire unit was assembled accordingly with the ladder (4-20% Tris-glycine SDS PAGE) and the entire test was carried out at 100 volts for 1 hour. Next, the gel was placed against the PVDF membrane, and sandwiched between filter papers and sponges where the electrophoretic transfer to the membrane at 100 voltages at cool temperature (0-4° C.) for 1 hour. After completing the electrophoretic transfer, the membrane was blocked with a blocking buffer (3-5% BSA in TBST or 5% milk) to avoid the non-specific binding sites, followed by washing with TBST (0.05% Tween 20 in Tris buffer, pH 7.4) for 3 times (each time 10 minutes); and incubated with primary antibody (Anti-Tau (phosphor Thr205) antibody, GTX24841) and Beta-actin (GTX629630) at 4° C. overnight. Next, preceded by 3 times wash with TBST and then an incubation with the secondary antibody (Goat anti-rabbit IgG (HRP), GeneTex and goat anti-mouse IgG (HRP)) was carried out, at ambient conditions for 1 hour. The membrane was reacted with an ECL kit (Western Lightning ECL Pro, Enhanced Chemiluminescence Substrate, P. Intertrade Equipment Co., Ltd., Khlongsan Bangkok, Thailand) by mixing 2 reagents in a 1:1 ratio. Finally, the signal (Chemiluminescence) was detected by Image Quant™ LAS 4000 (GEHealthcare, Chicago, IL, USA).

### 5. Elecrospining of PLGA-PLLA MTAMs

75:25 lactide: glycolide PLGA (MW 86,000) was purchased from Green Square Materials, Taiwan (P75DGOH065) and PLLA (MW 60,000) was purchased from Sigma (38534). Poly(ethylene glycol) (PEG) 35,000 (PEG-35 k) was purchased from Sigma (03557). Polyvinyl pyrrolidone (MW: 360 kDa) was purchased from Alfa Aesar (J61381). Dichloro-methane (DCM) and poly(ethylene oxide) (PEO) were purchased from Sigma (L021000 and 189,456 respectively). Materials were fabricated by adaptation of a core-shell co-axial microtube array membrane electrospinning technique. To prepare shell solution, PLGA, PLLA and porogen (PEG-35 k or PVP 360 kDa) were dissolved in DCM at 22-27% (w/v), wherein the ratio of PLGA and PLLA is 50:50 in weight. The emulsifier, PEG 40, was added (0.5% v/v) to the shell solution 5 minutes before electrospinning. To prepare core solution, PEG (35 kDa) and PEO (900 kDa) were dissolved in distilled water at a 1:1 ratio, with a concentration of 12-15% (w/v). The spinneret was moved longitudinally relative to the rotating collector and electrospinning was performed at 6.5±0.5 kV. The electrospinning process was conducted at 23±2° C. and 60±5% humidity. Shell solution and core solution flow rates were set between 4 and 12 mL/hour according to the viscosity. The material was soaked in distilled water for more than 24 hours to remove the core polymer and porogen, then dried. The final products were then cut into 0.5×2 cm pieces, treated with oxygen plasma (Harrick PDC-32G), and then UV-sterilized before use.

### 6. In Vivo Assay of PLGA-PLLA MTAMs

Establishment of an Alzheimer's disease (AD) cell model and Protein expression analysis of P-TAU.

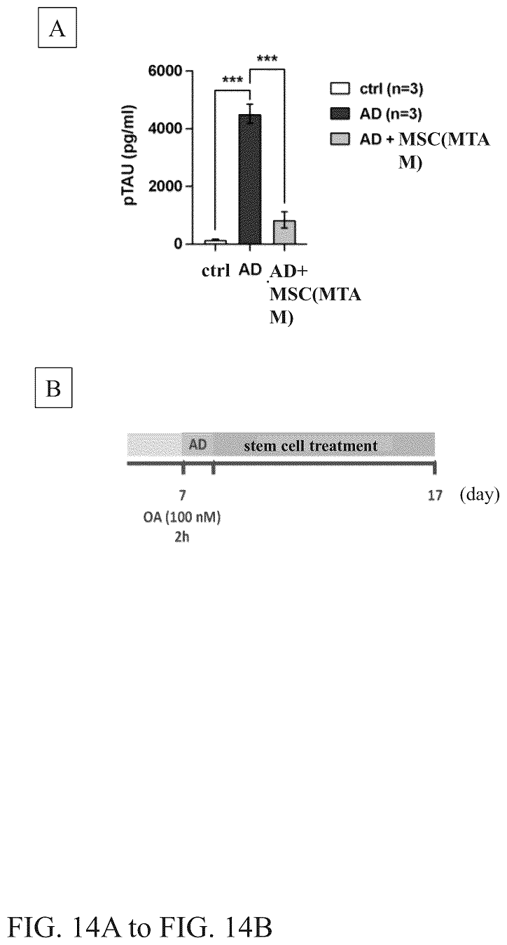

SH-SY5Y cells (neuroblastoma cell line) were differentiated into neuron-like cell by Retinoic acid and Brain-derived neurotrophic factor (BDNF), as control (ctrl). Referring to FIG. 14B, at day 7, neuron-like cells were induced into Alzheimer's Disease cell (AD cell) by Okadaic acid. AD cells were co-culture with MSC loaded in PLGA-PLLA MTAMs for 10 days (AD+MSC (MTAM)). Cell pellet of ctrl, AD, AD+MSC (MTAM) were collected for p-TAU ELISA kit (Elab, E-EL-H5314). Comparison (***p<0.001) is indicated by Tukey's post hoc test after one-way ANOVA.

### Nanoparticle Tracking Analysis (NTA)

2×10⁵ cells were loaded into 2×0.5 cm MTAMs, 5 MTAMs were put in one 25T flask with 5 mL medium. At the desired time point (day 7, 21, 28, 42, 56), 2.5 mL medium were collected for NTA analysis. Briefly, medium was centrifuged at 1000×g for 15 minutes to remove any possible cell debris, and the supernatant was collected and mixed with ExoQuick-TC at a 5:1 volume ratio. After treated at 4° C. for overnight, the medium/ExoQuick-TC mixture was centrifuge at 1500×g for 30 minutes. After centrifugation, the exosomes may appear as a beige or white pellet at the bottom of the vessel. The pellet was resuspended with 0.5 mL PBS (passed through a 0.22 μm filter) for the NTA analysis.

Specimens were analyzed using a NanoSight NS-300 (Malvern). The resuspended sample was diluted to a particle concentration ranging between 20 and 200 particles per frame. Each sample was measured three times for 60 seconds per measurement.

### 7. The Impact of the Implantation of PLGA-PLLA MTAMs in Mouse Models.

### Animal Behavior Test

### Novel Object Recognition (NOR) Test

The test was performed in a black plastic and black box (60×60×120 cm) with a camera on the top for behavior recording at 0-6 months of MSC (iv) or MSC (MTAM) treatment. MSC (iv) represents the treatment group receiving MSCs via intravenous injection, while MSC (MTAM) represents the treatment group receiving implantation of MSCs encapsulated in PLGA-PLLA MTAMs. The procedure included three phases: habituation, training, and testing. On day 1 to day 3 (habituation), a mouse was placed in the box for 10 minutes and allowed to freely explore the environment. On day 3, after habituation, each mouse was received 10 minutes of training in the box with two identical objects, and then it was returned to its home cage. Following a 30 minutes delay, the mouse was placed back into the box where it was presented one familiar object and one novel object for 10 minutes. Objects and the test area were cleaned with 70% ethanol after each task. The video was analyzed by Noldus software (Noldus, Leesburg, VA, USA). The amount of time spent on the novel object was compared to the time spent on the familiar object. Novel object preference scores were calculated as the percentage of total time spent on the novel object: NOR %=[(time of novel object)/(time of novel object)+ (time of familiar object)]×100).

APP/PSI mouse model (simulating Alzheimer's disease) and Mitopark mouse model (simulating Parkinson's disease) are both subjected to NOR test.

### Results

Preparation of Material for PSF MTAMs Loaded with Cell

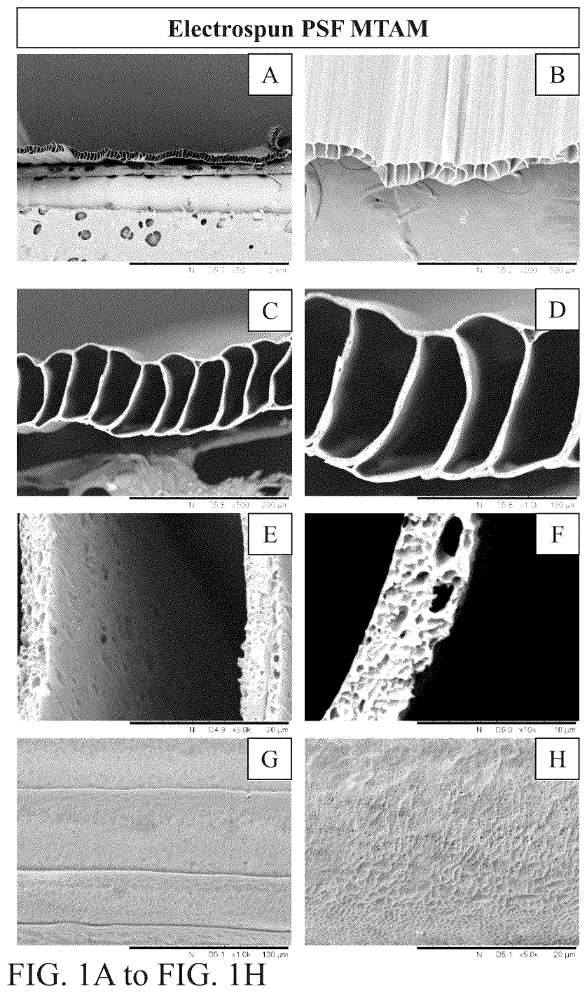

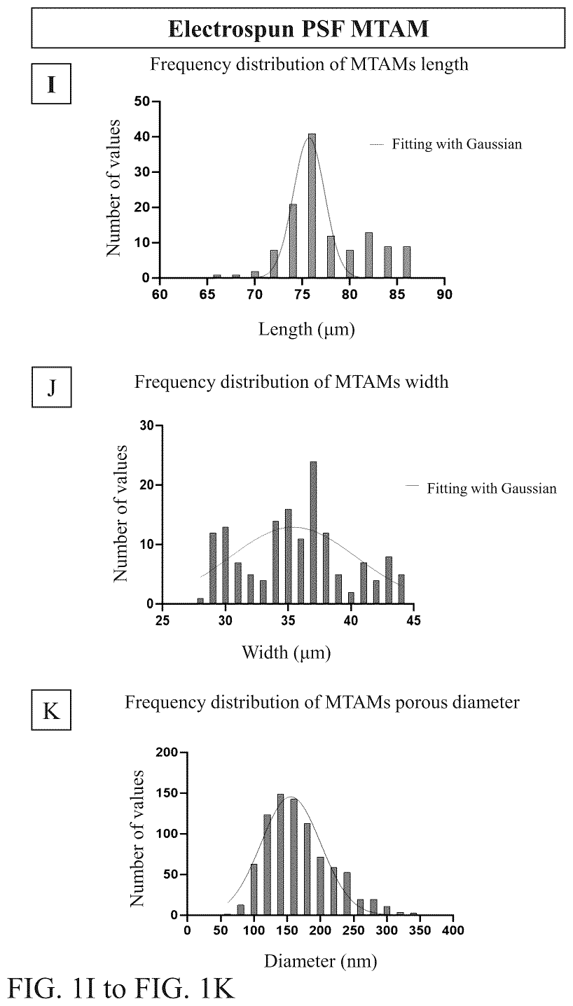

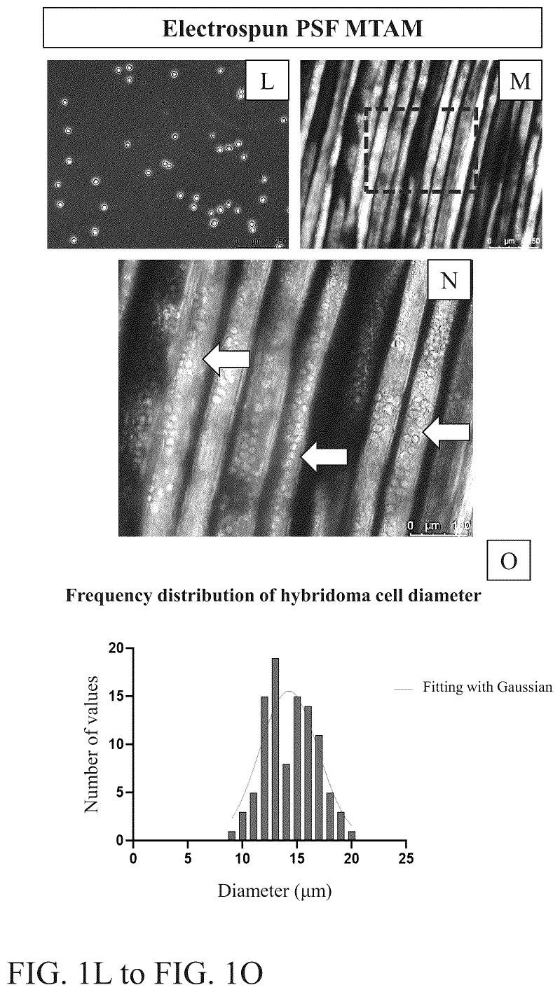

ImageJ quantification of the Scanning Electron Microscopy (SEM) images of the electrospun PSF MTAMs revealed a mean lumen dimension of 77.54±4.3 μm×35.64±4.2 μm (height×width). The lumen wall thickness of the individual lumen was 4.70±0.3 μm, while the pore size that were detected were around 167.75±50 nm (FIG. 1A to FIG. 1K). Majority of the distribution of these measurements were well within the Gaussian distribution curve.

As for the hybridoma, those cultured within the standard TCPs that were used as a reference, registering a mean dimension of 14.42±0.4 μm (n=6), as shown in FIG. 1O. These TCP cultured hybridoma proliferated well and the PSF MTAMs of 0.5 cm×1 cm could easily accommodate the entire hybridoma cell suspension of 2×10⁵ cells per 10 μL. Once siphoned into the respective PSF MTAMs, as shown in FIG. 1M and FIG. 1N, the cells could easily be observed by adjusting the focus of the optical microscopy. The siphoned hybridoma appeared to be evenly distributed across multiple individual lumens of the PSF MTAMs.

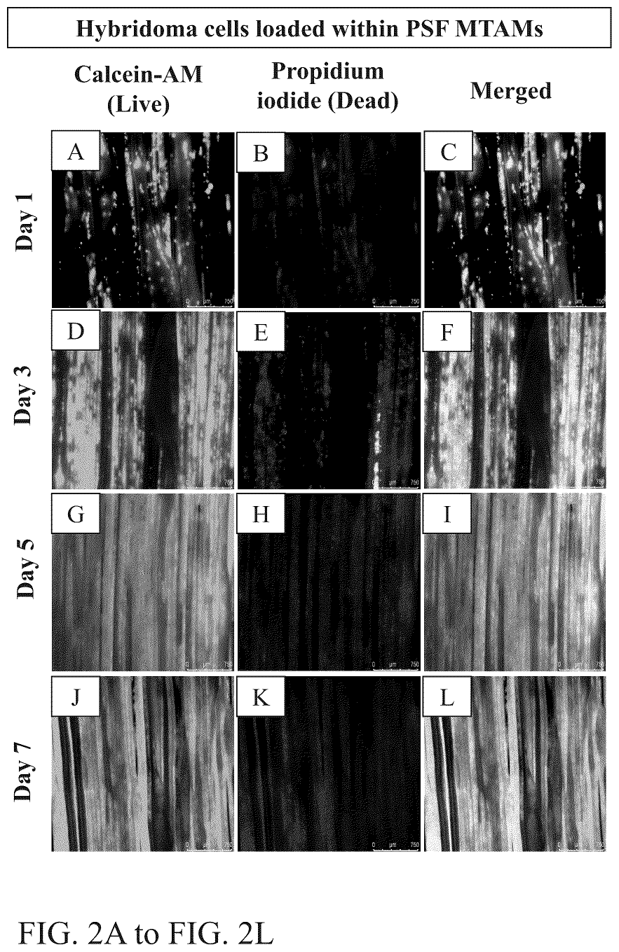

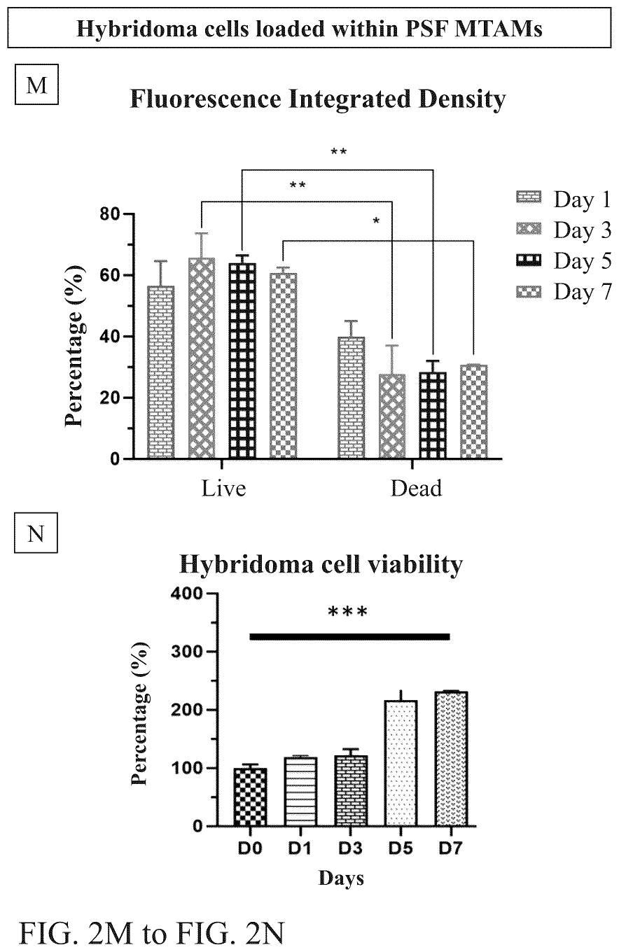

As shown in FIG. 2N, the cell viability of hybridoma were analyses by utilizing MTT assay, which revealed statistically significant increase between the readings on day 0 versus the viability on day 7. Two-way ANOVA with Sidak's multiple comparisons tests. *p<0.05, **p<0.01, ***p<0.001. Error bars in data represented +/−SD (n=3). With the passage of time, the cell proliferation progressed and began to plane off by day 5. By day 7, the overall hybridoma cell viability was statistically significantly higher than that at day 1. These observations were also corroborated in the Live-Dead Stain. As shown in FIG. 2D to FIG. 21, there was a significant increase in cell density between day 3 to day 5. At day 7, fluorescence integrated density also revealed a statistically significant higher live hybridoma. As shown in FIG. 2M, the total fluorescence integral density of the hybridoma cell line encapsulated in PSF MTAMs on day 7 revealed significantly higher live cells than the dead cells. Additionally, during the staining process, the respective dyes were able to be easily diffused into the lumens of the via highly porous surface, as illustrated in FIG. 1H.

The proliferation of hybridoma cell loaded within PSF MTAMs

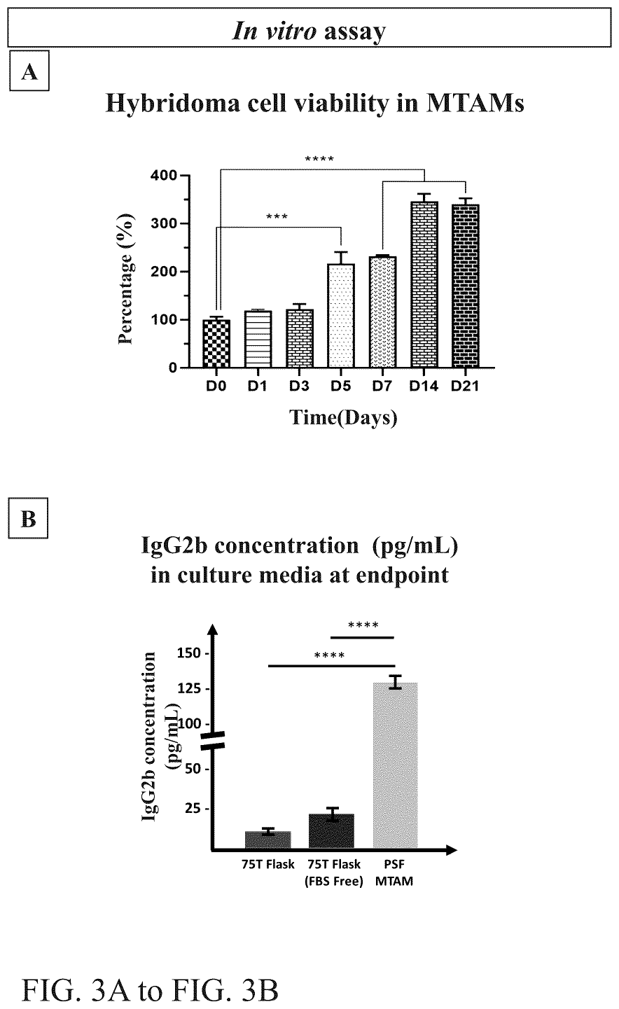

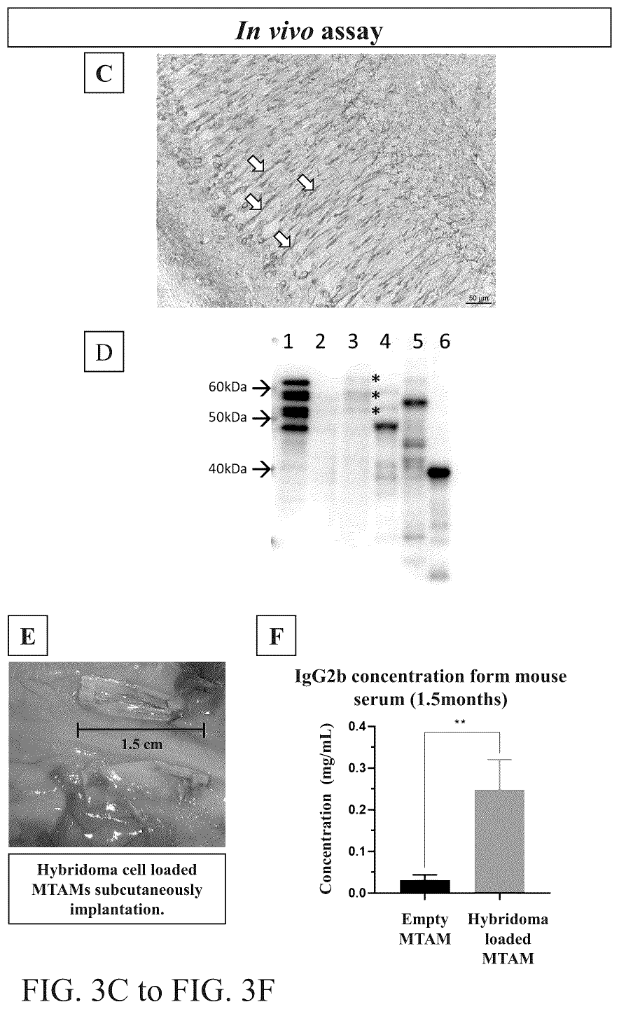

As shown in FIG. 3A, in vitro cell viability of hybridoma cells loaded within PSF MTAMs were quantified via MTT assay. At day 0, the cell density loaded within the PSF MTAMs were 2×10^{4/10} μL. At day 7, 14 and 21 the registered viability increased to 231±5%, 346±26%, and 340±21%, respectively. Both the short term (7 days, FIG. 2N) and long term (21 days, FIG. 3A) viability of the hybridoma cells encapsulated within the PSF MTAMs revealed significant increase in terms of viability. When compared to day 0, the viability of the hybridoma cells increased to 231±5% and 340±21% respectively; and this represents a statistically significant increase in terms of the overall viability. As shown in FIG. 3B, the functional release of antibodies (IgG2b) of hybridoma cells encapsulated within the PSF MTAMs registered significantly higher levels than those cultured within the standard 75T flasks at day 21. This number accounted for a 16-fold increase in functional antibody release of hybridoma cultured within PSF MTAMs as opposed to those cultured within the 75T flask.

The result of immunoblotting illustrates the in vitro cell survival rate of hybridoma cells loading in PSF MTAMs.

In another aspect of this embodiment, the ability of the hybridoma loaded PSF MTAMs to be implanted subcutaneously was also assessed. FIG. 3C illustrates the immunoreactivity of hybridoma supernatant (anti-tau IgG2b) in the pyramidale layer of the hippocampus in human tau (IN4R) transgenic mice. FIG. 3D indicates immunoblotting using of the hybridoma supernatant (anti-tau IgG2b) on different protein samples: 1: six recombinant tau isoforms (0N3R, 1N3R; 0N4R; 1N4R: 2N3R; 2N4R); 2: human healthy control brain homogenate: 3: Alzheimer's disease patient brain homogenate: note the typical tau triplet (asterisks): 4: mouse brain homogenate; 5: human tau (IN4R) transgenic mouse brain homogenate; 6: lysate of SY5Y neuroblastoma cell transfected with a fragment of tau protein (amino acids 1-265) indicating that the epitope is in the amino-terminal part of the protein. Molecular weight (arrows) is given on the left of the blot.

As shown in FIG. 3C and FIG. 3D, there were significantly difference in the serum of the host whereby, the IgG2b antibody levels where the mouse model with hybridoma loaded PSF MTAMs implanted registered an 8.3 fold higher levels of antibody compared to those implanted with empty PSF MTAMs.

Escape latency in the Morris Water Maze (MWM) for relative animal models.

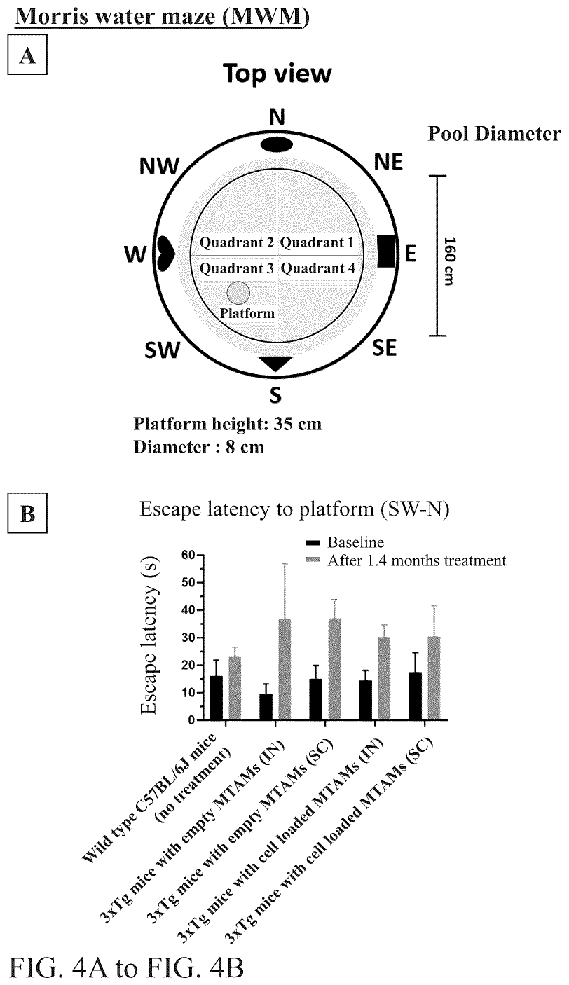

FIG. 4A is a design of the Morris water maze. The respective study groups were assessed with this system, namely wild-type (without treatment), intracranially (IN) implanted empty PSF MTAMs, subcutaneously (SC) implanted empty PSF MTAMs, IN implanted hybridoma loaded PSF MTAMs, and SC implanted hybridoma loaded PSF MTAMs. FIG. 4B is the escape latency of the respective mouse model study groups analyzed with Two-way ANOVA Tukey's multiple comparisons tests (Empty MTAMs vs cell loaded: IN group p-value=0.37: SC group p-value=0.58).

In FIG. 4B, the Morris Water Maze (MWM) test demonstrated the baseline escape latency for wild-type C57BL/6J mice was 16±15 seconds, while 3×Tg mice treated with empty MTAMs and hybridoma loaded in PSF MTAMs registered a baseline escape latency of 14±10 seconds in average. After 1.4 months treatment, 3×Tg mice treated with cell loaded MTAMs via both IN (30±12) and SC (31±22) were shorter than those treated with empty MTAMs. These results suggest better spatial learning and memory in 3×Tg mice treated with cell loaded MTAMs, indicating that treatment with both IN and SC implanted cell loaded MTAMs has a beneficial effect on cognitive function.

### Passive Avoidance Test for Mouse Model

During the assessment of long-term memory in the subsequent probing trial, as shown in FIG. 4C, the baseline in wild-type C57BL/6J mice registered a reading of 22±14 seconds, while the 3×Tg mice treated with empty and cell loaded PSF MTAMs registered 35±11 seconds in average. The 3×Tg mice treated with empty PSF MTAMs via IN or SC registered the baseline readings of 29±6 seconds and 34±12 seconds, respectively. After 1.4 months, a reduction in travel time was observed compare to the relative baseline, with readings of 19±3 s (IN) and 25±7 s (SC). As a result, after 1.4 months of treatment, the AD mouse model in both IN and SC groups spent less time in the goal quadrant compared to the relative baseline, indicating impaired spatial memory. However, when hybridoma cell loaded PSF MTAMs are implanted, the value registered in IN group after 1.4 months was 31±4 seconds, indicating a close value as compare to the relative baseline of 33±11 seconds. These similar values demonstrate the treatments with IN implanted hybridoma cell loaded PSF MTAMs improved spatial memory. Though the time spent in the goal quadrant for hybridoma cell loaded MTAMs SC implantation decreased from 46±10 seconds at baseline to 36±8 seconds after treatment.

During the assessment of short-term memory in the subsequent probing trial after 1.4 months of treatment, as depicted in FIG. 4D, the hybridoma cell loaded PSF MTAMs IN implantation group spent 31±17 seconds in the goal quadrant, which was higher than empty PSF MTAMs IN implantation (21±17 s). Similar results were also observed in SC implantation group, where the empty PSF MTAMs registered 28±9 seconds, compared to the SC implantation group with 33±12 seconds. This finding reinforces the notion that the treatment with implanted hybridoma cell loaded PSF MTAMs improved spatial memory in the AD mouse model, especially in the IN groups.

### Conditioning Test for Mouse Models

Conditioning test is one of the most widely used paradigms to assess learning and memory. In acquisition, firstly, the mice were allowed to explore in the two connected compartments. After exploration, the mice were likely to stay in the dark compartment instead of the light compartment. Next, the mice were given electric foot shock. If the mice remember that danger, the mice will rather stay in the light compartment than move to the dark side.

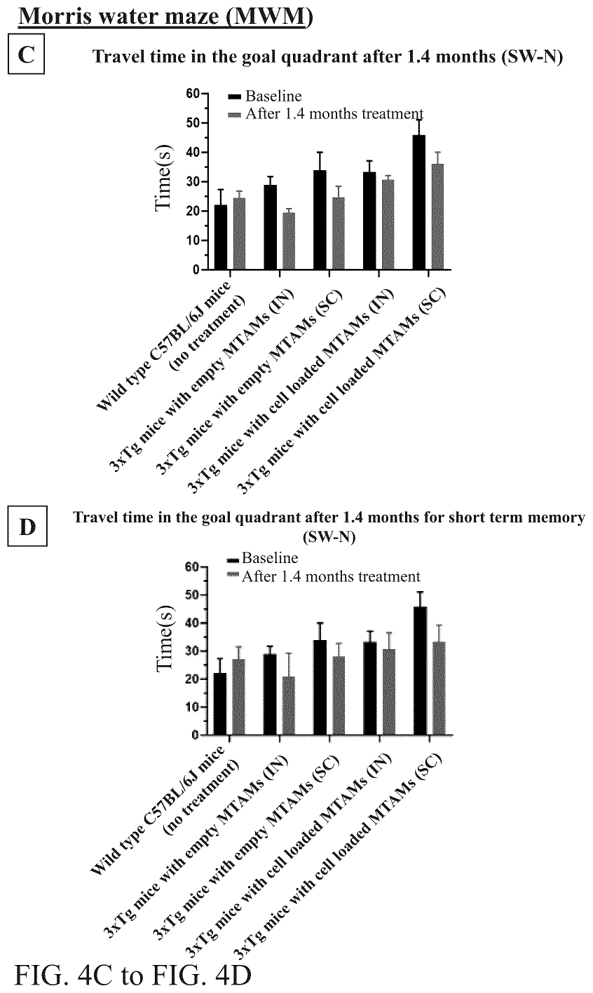

FIG. 5 shows the passive avoidance test results of the mouse models in each study group from day 1 to day 3 (baseline) and on day 46. Two-way ANOVA with Tukey's multiple comparisons tests. Significant impact p-value *p<0.05, **p<0.01. Error bars in data represented +/−SD. Wild-type, n=7, empty PSF MTAMs IN and SC, n=5 and n=2; hybridoma cell loaded PSF MTAMs IN and SC, n=7 and n=2.

As shown in FIG. 5, on day 3, the time for wild-type mice entered the dark compartment was 229±84 seconds, yet the time for 3×Tg mice entered the dark compartment was 61±103 seconds (baseline). After 1.5 months, on day 46, the step-through latency of hybridoma cell loaded PSF MTAMs IN group and SC group was 205±124 seconds and 190±112 seconds, respectively. Nevertheless, the step-through latency of empty PSF MTAMs intracranial implantation and subcutaneous implantation was 41±39 and 50±55 seconds. To ensure the reproducibility and reliability of results, we tested the empty PSF MTAMs and hybridoma cell loaded PSF MTAMs on further days (Day 46-48). Similarly, we gained the equivalent trend, comparing to the result of day 46. All the time units presented Mean±SD.

PSF MTAMs loading with hybridoma cell lines are capable of releasing IgG2b antibodies.

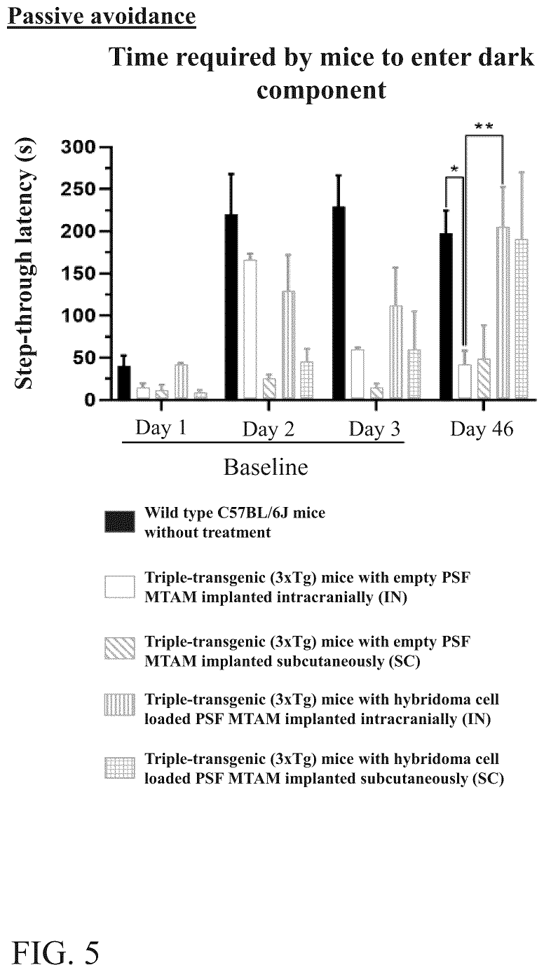

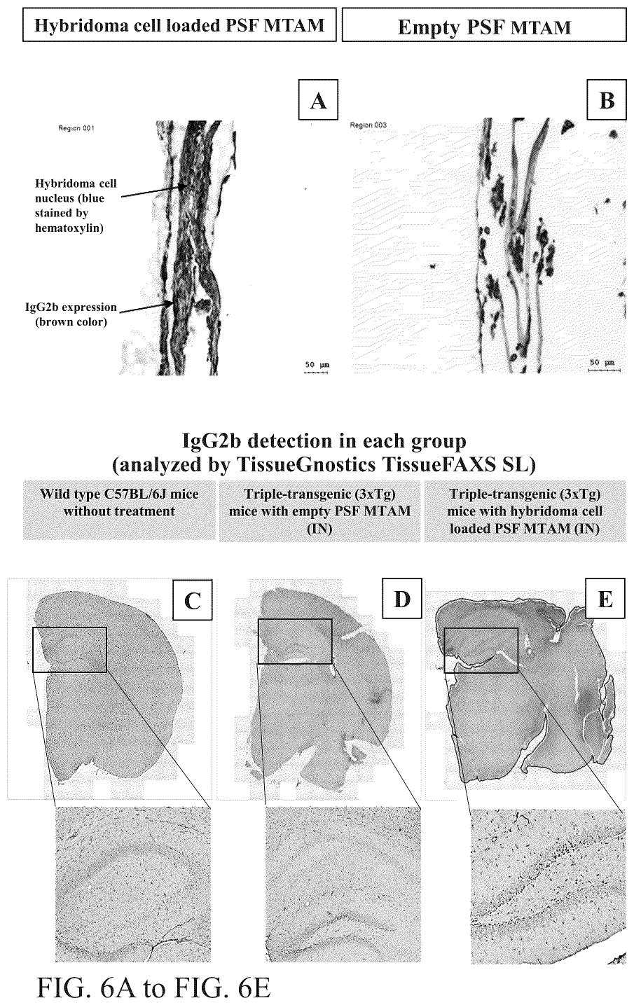

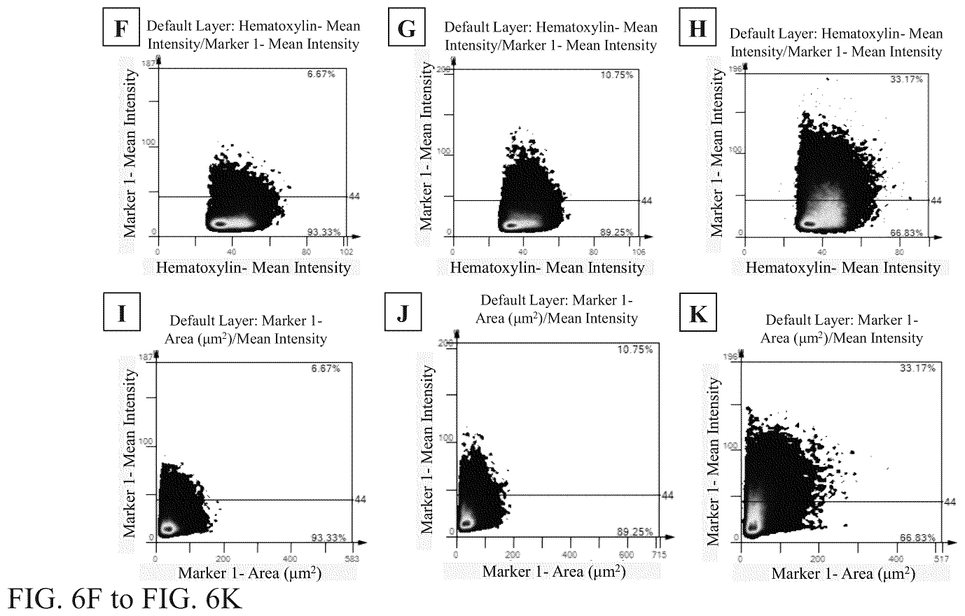

FIG. 6A illustrated immunohistochemistry of hybridoma cell loaded PSF MTAMs. The nucleus of the hybridoma cells were stained blue from the hematoxylin dye, while the IgG2b antibodies were stained brown. FIG. 6B, in contrast to FIG. 6A, shows a lack of blue/brown staining were observed within the empty PSF MTAMs.

Referring to FIG. 6A and FIG. 6B. In the immunohistochemistry staining of both empty PSF MTAMs and cell loaded PSF MTAMs, the cell loaded PSF MTAMs had a firm structure with hybridoma cells entrapped within PSF MTAMs. Moreover, the IgG2b expression was strong in the hybridoma cell loaded PSF MTAMs. The hybridoma cell's nucleus was stained by hematoxylin. However, the overall structure of empty PSF MTAMs appeared sparse. A weak IgG2b signal could be seen in empty PSF MTAMs. There were no live cells inside the empty PSF MTAMs.

FIG. 6C to FIG. 6E is a tissue section of the brain of mouse models. IgG2b antibodies appeared to be brown within nucleus, and blue when not within nucleus (counterstained by hematoxylin dye). FIG. 6C shows Wild-type C57BL/6J mice without treatment (n=5). FIG. 6D shows Triple-transgenic (3×Tg) mice with empty PSF MTAM (IN; n=3), and FIG. 6(E) shows Triple-transgenic (3×Tg) mice with hybridoma cell loaded PSF MTAM (IN: n=4), along with the corresponding magnified images.

FIG. 6F to FIG. 6H show the percentage of IgG2b positive signal in a single cell (y-axis) versus hematoxylin signals (x-axis), and FIG. 6I to FIG. 6K show the percentages of IgG2b positive signal in a single cell within each per IgG2b positive area.

FIG. 6L is a graph depicting the IgG2b positive signal in a single cell. FIG. 6M shows the corresponding IgG2b antibody distribution in total area.

To clarify whether IgG2b diffused into cortex and hippocampus by treating hybridoma cell loaded PSF MTAMs intracranially implanted directly on the brain surface, IHC was performed. IgG2b anti-tau antibodies were released by hybridoma cells, which were entrapped and proliferate in PSF MTAMs. As shown in FIG. 6L, the results indicated hybridoma cell loaded PSF MTAMs intracranial implantation, empty PSF MTAMs intracranial implantation, and wild-type mice had 18±10, 11±0.7, and 7±2% IgG2b positive area, respectively.

### Discussion

According to the ideal micro structure shown in FIG. 1A to FIG. 1H, the porous structure was observed in the morphology of PSF MTAMs. It suggested that the effect of blending PSF with Polyvinylpyrrolidone (PVP) exhibited phase inversion. Seeding the hybridoma cells in PSF MTAMs revealed that the PSF MTAMs was an excellent shelter to avoid rejection of immune response and can benefit anti-tau IgG2b antibody production. Briefly, this was primarily due to the sufficiently small pores which limited physical interaction between the host immune systems and the hybridoma cells encapsulated within, while allowing for nutrients, waste and signaling to pass freely across the lumen wall. Additionally, the semi translucent nature of the electrospun PSF MTAMs allowed for the easy observation of hybridoma cells encapsulated within PSF MTAMs via an optical microscope (FIG. 1M to FIG. 1N). As the hybridoma cells encapsulated within may potentially overlapping (when observed from above), the fine tuning of focus allowed us to adjust the plane of observation as illustrated in the above-mentioned figure.

In FIG. 2A to FIG. 2N, the overall survival of the hybridoma cells loaded within the PSF MTAMs revealed a significant increase between day 0 and day 7. Despite being significantly smaller than the standard 75T flask, the approximately 3× increase in viability seemed to indicate that the PSF MTAMs is an excellent substrate for cell culture, partly due to the biocompatible nature of the material used. The nano-topography which is conferred by the nano-pores, and the overall 3D configuration allowed for cell-to-cell interactions. The increase in viability continued until day 14 and remained sustained until day 21, as illustrated in FIG. 3A. In terms of antibody released from the hybridoma cell encapsulated within the PSF MTAMs (FIG. 3B), the amount of antibody released was significantly higher than those observed in the standard 75T flask. This observation indicated the microstructure and the close contact between hybridoma cells which allowed for cell-to-cell interaction does indeed trigger significantly higher functional release of antibody.

The diffusion of the released antibody into the blood of the host were examined. As shown in FIG. 3D, the hybridoma loaded PSF MTAMs when implanted revealed significantly higher levels of IgG2b antibody levels within the serum comparing to those implanted with the empty PSF MTAMs. This reinforced the notion that the highly, nano-porous surface of the MTAMs revealed for the diffusion of nutrient, waste and signaling molecules (antibodies in this case). While the diffusion of the antibodies across the lumen walls of the PSF MTAMs were not an issue, questions remained on the diffusion of these antibodies across the blood-brain barrier, which indirect evidence provided in FIG. 4A to FIG. 4D will be used for further discussion below.

In the Morris water maze (FIG. 4A to FIG. 4D), the 3×Tg mice with hybridoma cell loaded PSF MTAMs implanted (IN and SC) registered a significantly longer travel time in the goal quadrant and with shorter escape latency compared to study groups with empty PSF MTAMs implanted. This suggested that those implanted with hybridoma cell loaded PSF MTAMs did indeed registered an improvement in spatial learning and memory abilities in these transgenic mice models.

Passive avoidance test which is used to determine the learning and memory of the mouse model. As shown in FIG. 5, on day 46, the 3×Tg mice implanted with hybridoma cell loaded PSF MTAMs registered a statistically significantly higher step through latency when compared to those mouse models implanted with empty PSF MTAMs. This value was almost similar to those observed in the C57BL/6J mice models. This suggested that the hybridoma cell loaded PSF MTAMs do indeed significantly impact the outcome of Tau deposition in the corresponding area of the brain by releasing sufficient IgG2b antibodies, which also reduces neuro-inflammation, and ultimately affects the memory and learning of these hosts.

Referring to FIG. 5 and FIG. 6A to FIG. 6M, the results in behavioral test of the respective mouse models. suggested that when comparing the performance of mice with hybridoma cell loaded PSF MTAMs implanted intracranially, they exhibited significantly better performance than those implanted subcutaneously. Further studies from this point on was conducted on the IN site.

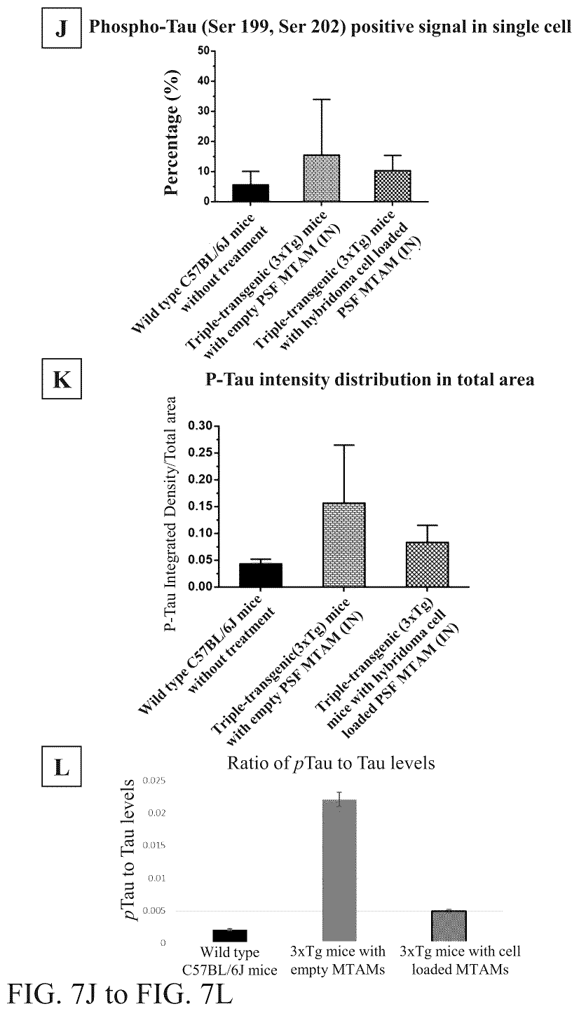

In FIG. 6A and FIG. 6B, the immunostaining of post implantation of the hybridoma cell loaded PSF MTAMs and the empty PSF MTAMs revealed significantly distinctive levels of IgG2b antibodies (brown staining) in and around the implanted hybridoma cell loaded PSF MTAMs (FIG. 6A). This finding reinforces the notion that these antibodies can freely diffuse from the hybridoma cells encapsulated within the PSF MTAMs to the surrounding tissues, blood stream and ultimately affects the target site—the brain of the host model. Furthermore, as shown in FIG. 6C to FIG. 6K, the tissue cross section analysis of the antibody deposition in the various regions of the host model's brains does further support the above-mentioned notion. Finally, the quantification of the IgG2b antibody in the brain of the mouse model shown in FIG. 8A and FIG. 8B revealed significantly higher levels for those study groups with hybridoma cell loaded PSF MTAMs, and this does indeed suggest the diffusion of the IgG2b antibodies to the respective regions of the brain.

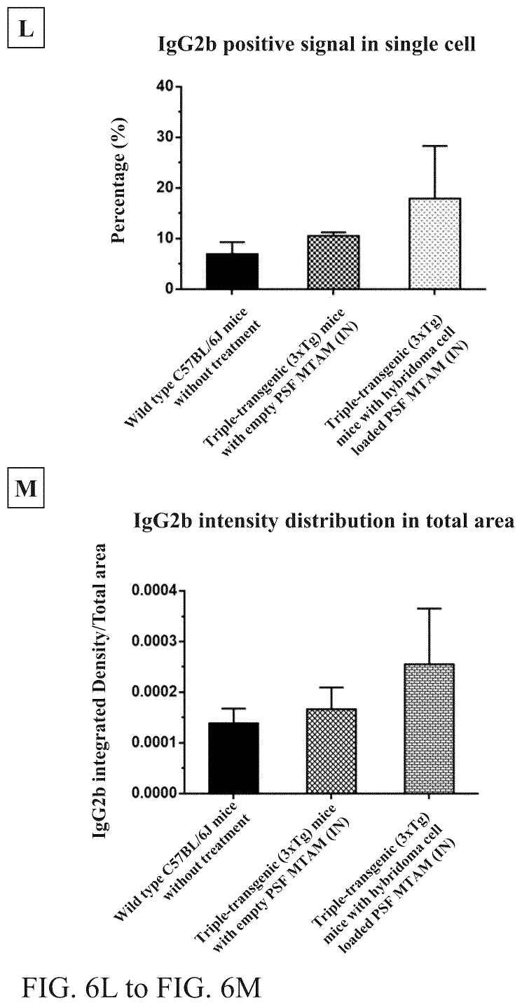

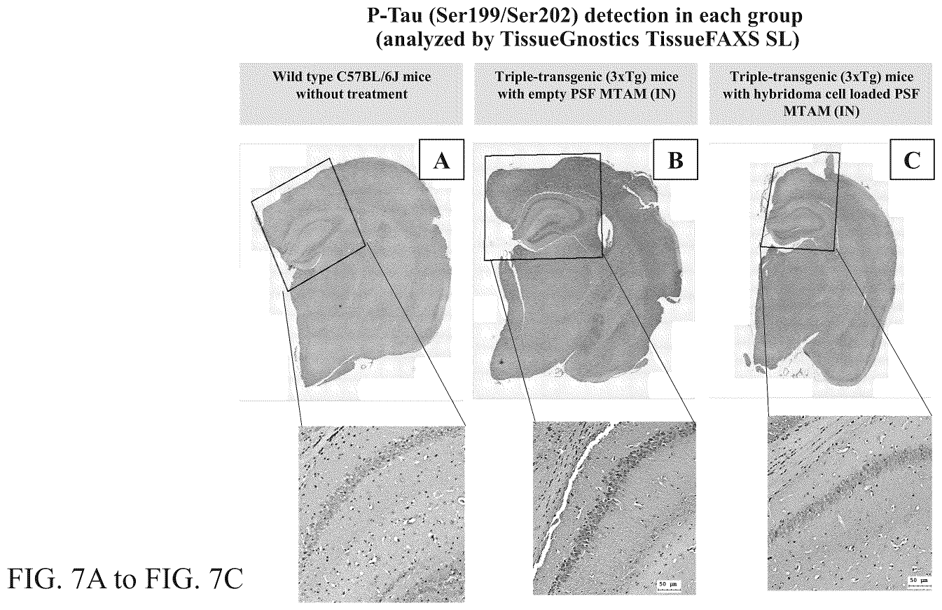

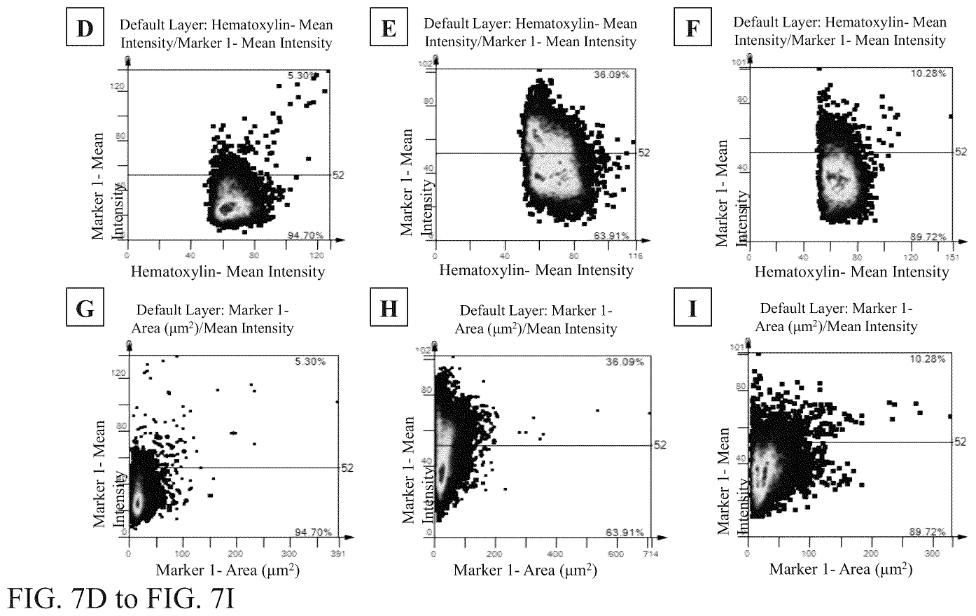

Regarding the distribution of the Tau protein in the mouse model's brain (FIG. 7A to FIG. 7L), an inversely correlated trend between the levels on IgG2b antibodies (FIG. 6L to FIG. 6M) and the P-Tau protein levels (FIG. 7J to FIG. 7L) of the mouse model's brain was observed, regardless of the brain tissue section or the final quantification of the Tau protein levels.

FIG. 7J is a graph depicting the P-Tau positive signal in a single cell. The Triple-transgenic (3×Tg) mice with empty PSF MTAM (IN) registering the highest percentage of 15 #11% as opposed to those in the wild-type C57BL/6J mice (6±3%) and Triple-transgenic (3×Tg) mice with hybridoma cell loaded PSF MTAMs (IN) (10±3%) respectively. FIG. 7K shows the corresponding IgG2b antibody distribution in total area. FIG. 7L shows the total Tau levels of the respective study groups.

FIG. 8A shows the western blot analysis of the respective study groups of the cortex and hippocampus regions targeting the P-Tau protein (79 kDa) and beta-actin (49 kDa). FIG. 8B depicts the ratio of P-Tau protein (79 kDa) after normalization with loading control. Triple-transgenic (3×Tg) mice with hybridoma cell loaded PSF MTAMs (IN) registered a P-Tau/Beta Actin loading control value in the cortex and hippocampus regions were 0.41±0.23 and 0.24±0.14, respectively. Conversely, the Triple-transgenic (3×Tg) mice with empty PSF MTAMs (IN) study group registered the highest values in the cortex and hippocampus regions were 0.48±0.17 and 0.51±0.11, respectively. While the wild-type C57BL/6J mice without treatment registered the lowest corresponding values in the cortex and hippocampus regions were 0.15±0 and 0.16±0.06.

As depicted in FIG. 7J and FIG. 7K, P-Tau protein levels were significant reduced in mouse models implanted with hybridoma cell loaded PSF MTAMs (IN) compared to those with empty PSF MTAMs, which the reduced levels were close to those observed in the wild-type mice. Also shown in FIG. 8A and FIG. 8B, the results suggested that the effect of the antibodies secreted by the encapsulated PSF MTAMs does indeed impact the overall distribution, deposition, and levels of P-Tau protein within the mouse model which ultimately positively impacted the memory, learning and behavioral patterns. In terms of the ratio of P-Tau to Tau levels which shown in FIG. 7L, it was found that the wild-type group registered the lowest reading, while the 3×Tg group with empty PSF MTAMs implanted registered the highest ratio. Such findings were in line with works by several groups which suggested that the underlying pathology of AD is the dysregulation of the balance between the P-Tau and normal Tau. Conversely, when 3×Tg groups were implanted with the hybridoma loaded PSF MTAMs, a significant reduction in the ratio was observed, thereby suggesting that the intervention with PSF MTAMs loaded with hybridoma cells restore a certain degree of this delicate balance.

### Preparing for the Encapsulated Cell in PLLA-PLGA MTAMs

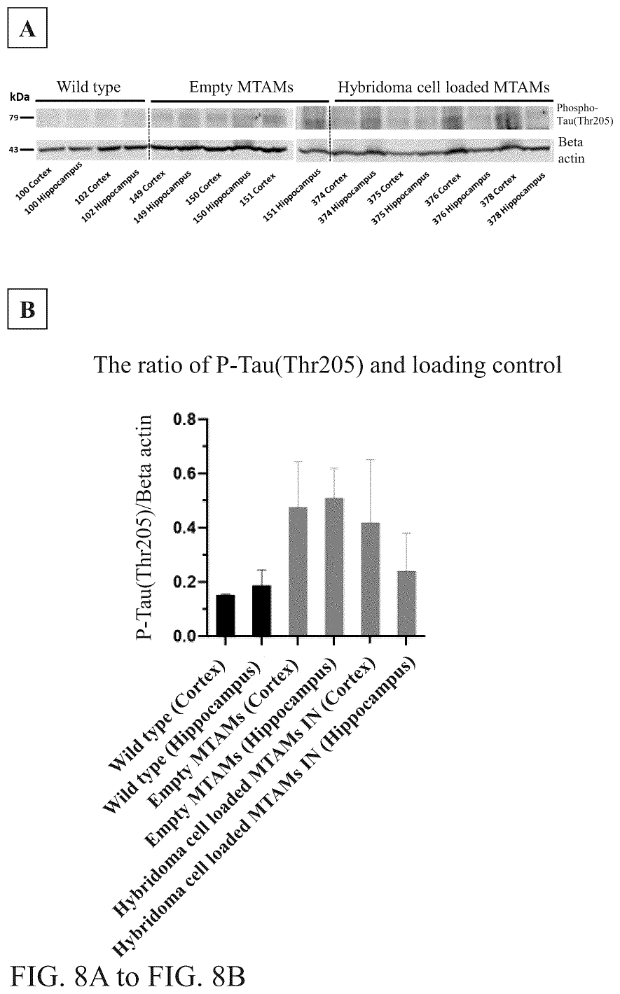

Referring to FIG. 9A to FIG. 9B for the SEM images of electrospun PLGA-PLLA MTAMs at magnification 200× and 5000×, respectively. The PLGA-PLLA MTAMs feature a wall thickness of 7.03±2.2 μm, along with a lumen length of 87.3±2.27 μm and a lumen width of 35.3±1.6 μm.

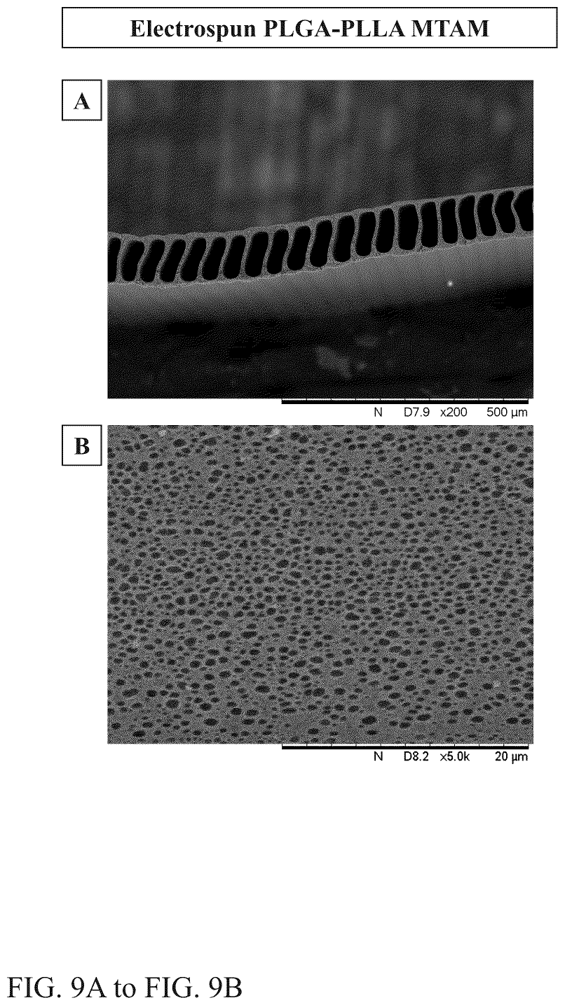

Referring to FIG. 10A to FIG. 10C, hUC-MCS cell (MSC), a TFDA approved stem cell, was provided by Meridigen. An attempt was made to determine whether MSCs can survive in MTAMs. Initially, 2×10⁵ MSCs are loaded in MTAMs, the viability of cells was determined by MTT assay. As shown in FIG. 10A, the OD values of MSCs were extremely low when MSCs loaded in PSF MTAMs, indicating that the cell cannot survive when cultured in PSF MTAMs. Subsequently, PLLA-PLGA MTAMs were used to culture human cardiac mesenchymal cells. As shown in FIG. 10B and FIG. 10C, the OD values of MSC were significantly higher, and remained stable until day 42, disclosured MSCs can survive well in this kind of MTAMs. Compared to loading in PSF MTAMs, hUC-MSC survives better in PLGA-PLLA MTAMs.

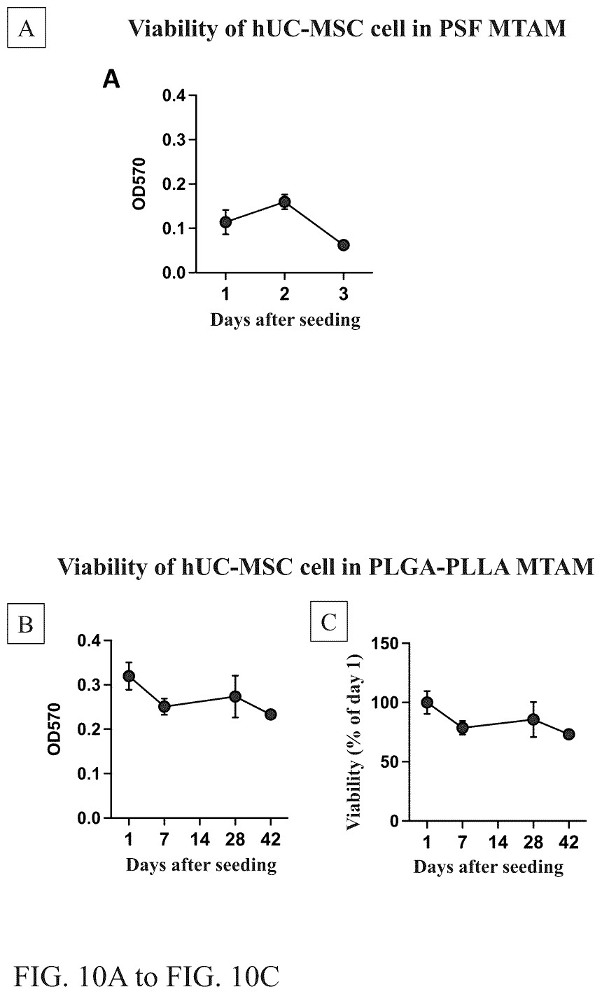

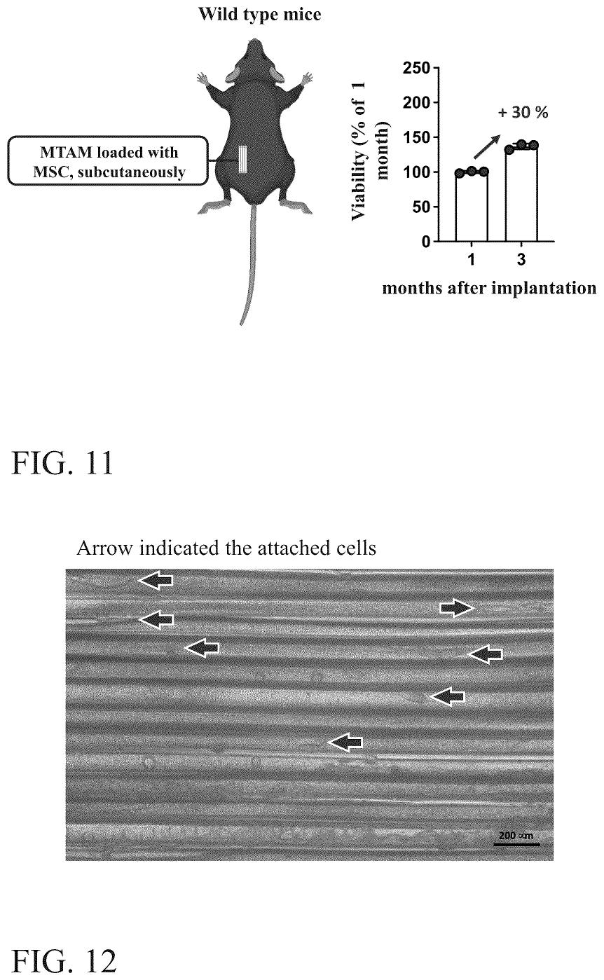

FIG. 11 shows the image and the viability of hUC-MSC loaded in PLLA-PLGA MTAMs, and FIG. 12 shows image of hUC-MSC loaded in PLLA-PLGA MTAM at day 28.

As shown in FIG. 11, three months after being implanted subcutaneously in animals, hUC-MSC encapsulated in PLGA-PLLA MTAMs increased the survival rate of the stem cells at about 30%. This observation suggests that stem cells can not only survive but also proliferate when cultured under these conditions, suggesting a favourable environment for their growth. As shown in FIG. 12, the arrows in the figure refer to cells with a spindle-shaped appearance and normal attachment, indicating that the cells can proliferate and maintain viability inside PLGA-PLLA MTAMs.

### Stem Cells Encapsulated in PLGA-PLLA MTAMs Shown Normal Function

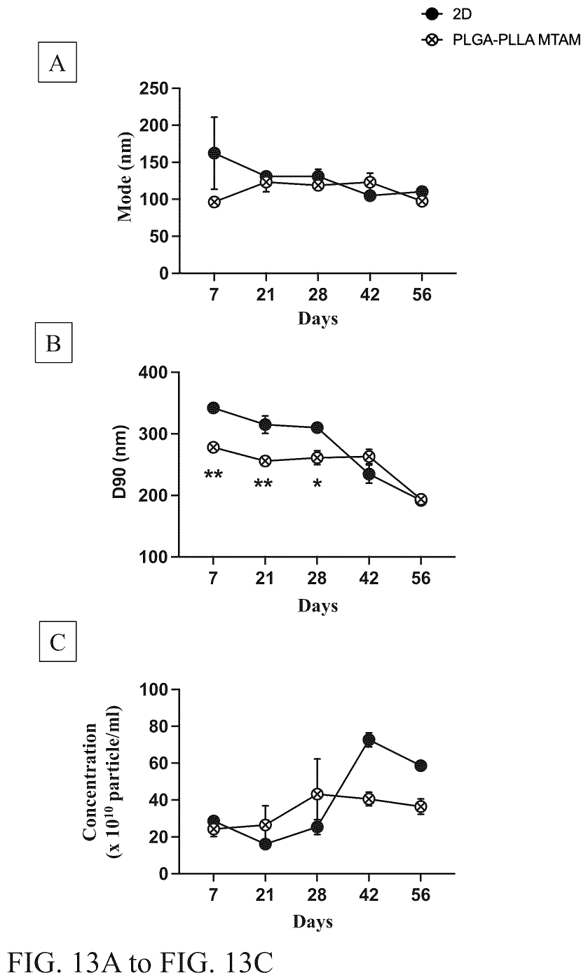

Particles were released from stem cells, the trend of the particle released from stem cells is shown in FIG. 13A to FIG. 13C. Half of the total medium volume was analysed at each time point, wherein, the dots marked with x (\textcircled{X}) represent that at Day1, 2×10⁵ MSCs were loaded in PLGA-PLLA MTAMs, and 5 MTAMs were placed in a 25T flask. The black dots (●) indicated 106 MSCs were directly cultured in a 75T flask in 2D manner. FIG. 13A shows the diameter distribution of sample particles. FIG. 13B, D90 (nm) indicates the mean of the highest 90% diameter in the sample at each time point. *p<0.05, **p<0.01 (PLGA-PLLA MTAM compared with 2D-cultured at each time point) are defined by Tukey's post hoc test after two-way ANOVA. In FIG. 13C, “concentration” refers to the particle concentration measured at each time point.

The MTAMs possess a pore size within the range of 100-900 nm. NTA results shown the release of particles originating from stem cells which correspondingly emanate from MTAMs.

In FIG. 14A, pTAU ELISA was performed after AD cells co-culture with MSCs, wherein, Ctrl denotes the control group, namely healthy non-AD cells. AD denotes the untreated disease cells. AD+MSC (MTAM) represents AD cell treated with MSCs encapsulated in PLGA-PLLA MTAMs. The results shown that the protein expression of p-TAU is reduced in Alzheimer's disease (AD) cells after co-culture with hUC-MSC.

Recovery of recognition memory in mice through MSC (MTAMs) treatment

During the testing phase of the NOR test, the environment where the mice were placed with both the old objects (old), which the mice had encountered 10 minutes prior, and the new objects (new), which the mice had not previously encountered. The purpose of the NOR test is to observe whether the mice can distinguish between old and new objects, thereby determining their cognitive ability. If the mice perceive the object as new, they will exhibit increased exploration and olfactory investigation by wandering around and smelling it. This behaviour of the mice can be inferred through the observation of their movement trajectory with longer cumulative duration and increased interaction frequency with the objects. If there is an increase in the interaction frequency or cumulative duration with the new object compared to the disease control group after treatment, it can be indicated that there is an improvement in cognitive function and that the treatment is effective.

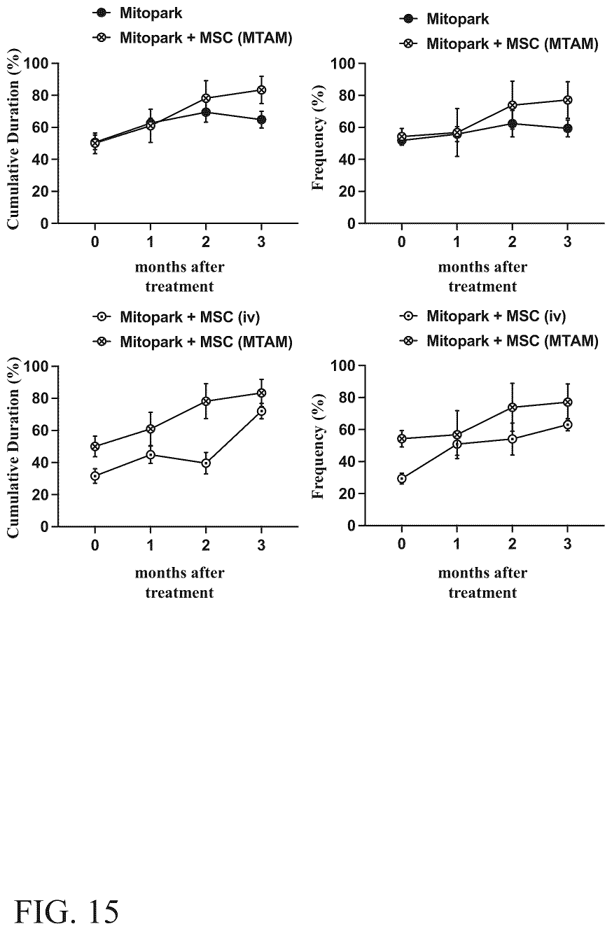

Referring to FIG. 15, the restoration of recognition memory function in mice following MSC (MTAM) treatment after 3 months is assessed through novel objects recognition (NOR) test. Mitopark is an animal model simulating Parkinson's disease. At 0, 1, 2, and 3 months after MTAMs implantation, compared to PD control group (labelled with Mitopark), both the interaction frequency and the cumulative duration with the new object increased in mice implanted with PLGA-PLLA MTAMs. The results indicated that the treatment with MSC (MTAM) can aid in the recovery of recognition memory in Parkinson's disease mice after 3 months. The therapeutic effect of MSC (MTAM) is superior to that of intraperitoneal injection of MSC (indicated by the MSC (iv) group). This demonstrates that MSC (MTAM) possess therapeutic effects, exhibiting improvement in memory in animal models.

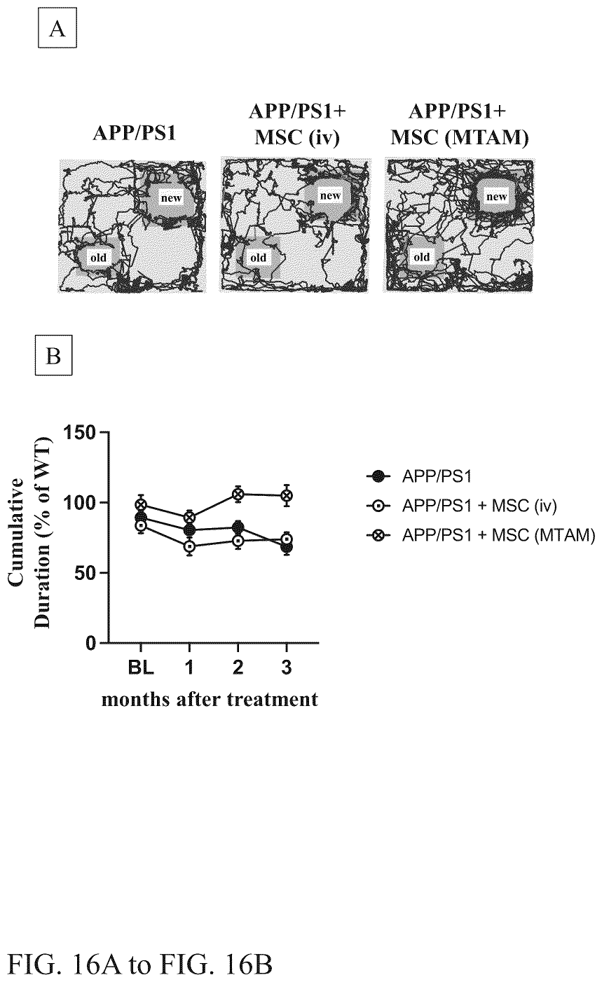

FIG. 16A to FIG. 16B shows the restoration of recognition memory in APP/PSI mice after 3 months of treatment with MSCs encapsulated in PLGA-PLLA MTAMs. APP/PSI is an animal model simulating Alzheimer's disease. Using a similar experiment as in FIG. 15, FIG. 16A shows the recordation of the representative motion traces of each group within 10 minutes. FIG. 16B represents the calculation of the percentage of time spent on the new object. The data are presented as the mean±SEM. Both the traces and the data were obtained by EthoVision XT. The results show a similar trend as in FIG. 15, indicating that MSC (MTAM) has therapeutic effects and aids in the restoration of recognition memory in Alzheimer's disease mice.

In summary, according to the present disclosure, the beneficial properties of functional cells encapsuled in MTAMs are as follows:

Unhindered diffusion of therapeutic biological products secreted by cells:

Selective diffusion of small sized exosomes carrying therapeutic factors, while limiting larger exosome carrying pro-apoptotic factors.

Functional cells cultured under 3D conditions, with good cell-to-cell contact resulting in significant improvement in long term functional viability.

Immune cell attacks blocked physically by the encapsulating MTAM.

What is claimed is:

1. A pharmaceutical composition for treating neurodegenerative diseases of the brain, wherein the pharmaceutical composition comprises cells encapsulated by microtubular array membranes (MTAMs), the cells comprise hybridoma cells capable of secreting anti-Tau antibodies or stem cells.

2. The pharmaceutical composition according to claim 1, wherein the type of stem cells comprises neural stem cells (NSCs), umbilical cord stem cells, amniotic fluid stem cells (AFSCs), bone stem cells, adipose stem cells, or umbilical cord tissue-derived mesenchymal stem cells (hUC-MSCs).

3. The pharmaceutical composition according to claim 1, wherein type of the stem cells is umbilical cord tissue-derived mesenchymal stem cells (hUC-MSCs).

4. The pharmaceutical composition according to claim 1, wherein the neurodegenerative disease comprises Alzheimer's disease or Parkinson's disease.

5. The pharmaceutical composition according to claim 1, wherein a material of the microtubular array membrane is Polysulfone (PSF) or a copolymer of Poly(lactic-co-glycolic acid) and Poly-L-lactic acid (PLGA-PLLA).

6. The pharmaceutical composition according to claim 1, wherein the microtubular array membrane has fibers being arranged along an extension direction.

7. The pharmaceutical composition according to claim 1, wherein the microtubular array membrane is a highly aligned and densely packed electrospun fibers assembly, the fibers are packed together to form a monolayer, and the orientation of the fibers is not greater than +/−5° relative to a longitudinal axis of the assembly.

8. The pharmaceutical composition according to claim 1, wherein an average lumen size of the microtubular array membrane ranges from 80 μm to 120 μm in height and from 40 μm to 60 μm in width.

9. The pharmaceutical composition according to claim 1, wherein the microtubular array membrane has hollow fibers arranged in a monolayer array.

10. The pharmaceutical composition according to claim 1, wherein a wall thickness of a single lumen of the microtubular array membrane ranges from 3.5 μm to 8 μm.

11. The pharmaceutical composition according to claim 1, wherein a pore size of the microtubular array membrane ranges from 100 nm to 900 nm.

12. The pharmaceutical composition according to claim 1, wherein an average diameter of cells encapsulated in the microtubular array membrane ranges from 12 μm to 18 μm.

13. A method for treating neurodegenerative diseases of the brain in a subject in need thereof, comprising implanting the pharmaceutical composition according to claim 1 into a living body.

14. The method according to claim 13, wherein the step of implanting comprises intracranial implantation or subcutaneous implantation.

15. The method according to claim 13, wherein the neurodegenerative disease comprises Alzheimer's disease or Parkinson's disease.
